SEQUENCE LISTING (31 sequences)

SEQ ID NO: 1 - protein, 12 aa, Homo sapiens
  MISC_FEATURE (1)..(12): Sequence may include an amino terminal capping group and/or a carboxy terminal capping group
QYKLGSKTGPGQ

SEQ ID NO: 2 - protein, 6 aa, Homo sapiens
  misc_feature (1)..(6): Sequence may include an amino terminal capping group and/or a carboxy terminal capping group
QTLQFR

SEQ ID NO: 3 - protein, 7 aa, Homo sapiens
  MISC_FEATURE (1)..(7): X at position 1 is Asp or Asn; X at position 3 is Asp or Asn; X at position 4 is absent or Gly; X at position 5 is absent, Asp, or Phe; X at position 6 is absent, Ala or Phe; X at position 7 is absent or Ala
  MISC_FEATURE (1)..(7): Sequence may include an amino terminal capping group and/or a carboxy terminal capping group
XGXXXXX

SEQ ID NO: 4 - protein, 5 aa, Homo sapiens
DGDGD

SEQ ID NO: 5 - protein, 6 aa, Homo sapiens
DGDGFA

SEQ ID NO: 6 - protein, 7 aa, Homo sapiens
DGDGDFA

SEQ ID NO: 7 - protein, 7 aa, Homo sapiens
DGNGDFA

SEQ ID NO: 8 - protein, 7 aa, Homo sapiens
NGNGDFA

SEQ ID NO: 9 - protein, 7 aa, Homo sapiens
NGDGDFA

SEQ ID NO: 10 - protein, 8 aa, Homo sapiens
  MISC_FEATURE (1)..(8): Sequence may include an amino terminal capping group and/or a carboxy terminal capping group
  MISC_FEATURE (1)..(2): X at position 1 is absent or is Ser; X at position 2 is absent or is Lys
XXMTLTQP

SEQ ID NO: 11 - protein, 6 aa, Homo sapiens
MTLTQP

SEQ ID NO: 12 - protein, 8 aa, Homo sapiens
SKMTLTQP

SEQ ID NO: 13 - protein, 5 aa, Homo sapiens
  MISC_FEATURE (1)..(5): X at position 3 is Glu or Leu; X at position 4 is Ala or Glu; X at position 5 is absent, Leu or Ala
  MISC_FEATURE (1)..(5): Sequence may include an amino terminal capping group and/or a carboxy terminal capping group
DGXXX

SEQ ID NO: 14 - protein, 4 aa, Homo sapiens
  MISC_FEATURE (1)..(4): Sequence may contain an amino terminal capping group and/or a carboxy terminal capping group
  MISC_FEATURE (1)..(): An acetyl amino terminal capping group may be appended to the sequence
DGEA

SEQ ID NO: 15 - protein, 11 aa, Homo sapiens
  MISC_FEATURE (1)..(7): X in the first position is absent or any amino acid; X in the second position is absent or any amino acid; X in the fifth position is Glu or Leu; X in the sixth position is Ala or Glu; X in the seventh position is absent, Leu or Ala
  MISC_FEATURE (1)..(11): Sequence may contain an amino terminal capping group and/or a carboxy terminal capping group
  MISC_FEATURE (8)..(11): X in the eighth position is absent or any amino acid; X in the ninth position is absent or any amino acid; X in the tenth position is absent or any amino acid; X in the eleventh position is absent or any amino acid
XXDGXXXXXXX

SEQ ID NO: 16 - protein, 5 aa, Homo sapiens
  misc_feature (1)..(5): Sequence may contain an amino terminal capping group and/or a carboxy terminal capping group
DGEAL

SEQ ID NO: 17 - protein, 5 aa, Homo sapiens
DGLEA

SEQ ID NO: 18 - protein, 6 aa, Homo sapiens
ETLQFR

SEQ ID NO: 19 - protein, 8 aa, Homo sapiens
QYSIGGPQ

SEQ ID NO: 20 - protein, 8 aa, Homo sapiens
SDRSARSY

SEQ ID NO: 21 - protein, 12 aa, Homo sapiens
DGDGDFAIDAPE

SEQ ID NO: 22 - protein, 5 aa, Homo sapiens
NGNGD

SEQ ID NO: 23 - protein, 5 aa, Homo sapiens
DGNGD

SEQ ID NO: 24 - protein, 5 aa, Homo sapiens
NGDGD

SEQ ID NO: 25 - protein, 4 aa, Homo sapiens
NGDG

SEQ ID NO: 26 - protein, 6 aa, Homo sapiens
DGDGFA

SEQ ID NO: 27 - protein, 6 aa, Homo sapiens
NGNGFA

SEQ ID NO: 28 - protein, 6 aa, Homo sapiens
DGNGFA

SEQ ID NO: 29 - protein, 6 aa, Homo sapiens
NGDGFA

SEQ ID NO: 30 - DNA, 18 bp, Homo sapiens
aatccgtcgagcagagtt

SEQ ID NO: 31 - DNA, 18 bp, Homo sapiens
ctaaccctaaccctaacc

(19) United States
(12) Patent Application Publication
(10) Pub. No.: US 2007/0042962 A1
(43) Pub. Date: Feb. 22, 2007

(54) Peptide dependent upregulation of telomerase expression

(76) Inventors: David S. Adams, Worcester, MA (US); Victor E. Shashoua, Brookline, MA (US)

Correspondence Address: PATENT DOCKET ADMINISTRATOR;LOWENSTEIN SANDLER PC, 65 LIVINGSTON AVENUE, ROSELAND, NJ, 07068, US

(21) Appl. No.: 10/511,530

(22) PCT Filed: Feb. 3, 2003
(86) PCT No.: PCT/US03/03425

§ 371 Date: Jul. 6, 2005

Related U.S. Application Data
(60) Provisional application No. 60/354,423, filed on Feb. 4, 2002.

(51) Int. Cl. A61K 38/10; A61K 38/08; A61K 38/05

(52) U.S. Cl. 514/15; 514/16; 514/17; 514/18

(57) ABSTRACT

Methods and compositions comprising peptide compounds are described to upregulate expression of telomerase in eukaryotic cells, tissues, and organs. Such methods and compositions are particularly useful in treating diseases, trauma, and conditions of the aging process.

### BACKGROUND

Telomeres are nucleoprotein structures located at the end of eukaryotic chromosomes that contain protein-bound, simple repeat units of a nucleotide sequence (Rhyu, J. Natl. Cancer Inst., 87:884-894 (1995)). Telomeres protect chromosomes from shortening and unraveling during each replication cycle. The function of telomeres has been compared to the role of metal or plastic ends of shoelaces.

Telomeres have been studied in a variety of eukaryotic organisms. For example, Tetrahymena contains up to 40,000 telomeres per DNA macromolecule, each containing the repeat sequence GGGGTT (Blackburn and Gall, J. Mol. Biol., 120: 33-53 (1978)). Telomeres of many insects, including crickets, cockroaches, and Lepidopteran species, contain the pentanucleotide repeat sequence TTAGG (Sasaki and Fujiwara, Eur. J. Biochem., 267(10): 3025-3032 (2000)). In the diploid human cell there are 46 chromosomes, each containing two telomeres, and each human telomere contains the nucleotide repeat sequence TTAGGG, which may be repeated up to 15 kilobases (kb) per telomere (Moyzis et al., Proc. Natl. Acad. Sci. USA, 85: 6622-6626 (1988)). With the exception of germ line cells, such as sperm, the telomere repeat unit in human cells gradually decreases over time by approximately 15-40 base pairs per year per somatic cell (see, e.g., Allsopp et al., Proc. Natl. Acad. Sci. USA, 89: 10114-10118 (1992); Hastie et al., Nature, 346: 866-868 (1990)). Once telomeres reach a certain short length, cell division (mitosis) halts, the cell enters a state known as senescence (i.e., the cell ages without division), and eventually dies. Telomeres are made and replenished by the action of the enzyme telomerase, which is a complex nucleoprotein enzyme comprising a reverse transcriptase subunit (designated “TERT”) and an RNA component (designated “TR”). Human genes encoding the protein component (hTERT gene) and the RNA component (hTR gene) for telomerase have been previously cloned and sequenced (regarding TERT gene, see, Nakamura et al., Science, 277: 955-959 (1997); Kilian et al., Human Mol. Genetics, 6: 2011-2019 (1997); Takakura et al., Cancer Res., 59: 551-557 (1999); Wick et al., Gene, 232: 97-106 (1999); Cong et al., Human Mol. Genetics, 8: 137-142 (1999); regarding hTR gene, see, Feng et al., Science, 269: 1236-1241 (1995)). The RNA component of telomerase is reverse transcribed by TERT into the telomere DNA sequence attached to the ends of each linear chromosome in the nucleus of a eukaryotic cell. Telomerase is inactive (i.e., very low or undetectable) in most normal somatic tissue; hepatocytes and activated T cells are notable exceptions to this general rule.

From the documented loss over time of telomere sequences in somatic cells as well as other observations on telomere length, there has emerged the concept that sustaining and replenishing telomeric sequences is specific to the germ line (sperm and oocytes). Thus, Allsop et al. (Proc. Natl. Acad. Sci. USA, 89: 10114-10118 (1992)) measured telomere lengths from fetal tissue and tissue from adults up to 93 years of age and found that telomere length correlated strongly with the replicative ability of cells. Furthermore, the number of cell divisions that could take place in cultures was directly correlated to the initial length of the telomeres present at the time a tissue sample was isolated and started in culture. Allsop et al. (Proc. Natl. Acad. Sci. USA, 89: 10114-10118 (1992)) also showed that cell samples from individuals with the premature aging disease Hutchinson-Gilford progeria contained telomeres with a significantly shorter average length than telomeres from normal donors of the same age. Accordingly, the gradual depletion (i.e., shortening) of chromosome telomeres in somatic cells has been correlated with the phenomenon of aging, including loss of structural and metabolic components and functions necessary to maintaining healthy functioning cells, tissues, and organs (see, e.g., Hastie et al., Nature, 346: 866-868 (1990)).

Telomeres also play a critical role in programmed cell death (apoptopsis) and normal functioning tissues and organs. For example, telomerase-deficient mice, which lack a function mouse TR gene, have been generated in which the absence of telomerase expression in the cells, tissues, and organs of such animals was correlated with a significant acceleration or increase in genomic instability, impaired cell proliferation, and apoptosis in organ systems, for example, as evidenced by development of cirrhosis of the liver (see, e.g., Lee et al., Nature, 392: 569 (1998); Rudolph et al., Cell, 96: 701 (1999)). More recently, it was shown that a functional mouse TR gene could be restored in the liver cells of such telomerase-deficient mice using an adenoviral vector, resulting in the restoration of telomerase activity and telomere function, the alleviation of cirrhotic pathology, and the improvement in liver morphology and function (Rudolph et al., Science, 287: 1253-1258 (2000)). This study also documented that loss of telomerase activity and shortening of telomeres may be accompanied at the genetic level by an increase in chromosome abnormalities, including chromosome bridges and chromosome malformations. The concept that decreased stabilization of telomeres (i.e., decrease in telomere length and/or number) is related to cellular immortality was established from studies of various types of tumors (see, e.g., Hastie et al., Nature, 346: 866-868 (1990); Adamson et al., Cancer Genzet. Cytogeizet., 61: 204-206 (1992); Odagiri et al., Cancer, 73: 2978-2984 (1994); Rogalla et al., Cancer Genet. Cytogent., 77: 19-25 (1994); Shirotani et al., Lung Cancer, 11: 2941 (1994); and Yamada et al., J. Clin. Investig., 95: 1117-1123 (1995)). In general, telomeres were found to be notably shorter in immortal cells than those in mortal tissues. An exception to this correlation appears to be the immortal HeLa cell line from a uterine cervical carcinoma that contains exceptionally long telomeres (de Lange et al., Mol. Cell Biol., 10: 518-527 (1990)).

In general, telomerase levels are relatively high (upregulated) in progenitorial cells and neurons during early development and decrease in association with cell differentiation (see, e.g., Mattson et al., J. Neurosci. Res., 63(1): 1-9 (2001)). Upregulation of telomerase expression for telomere maintenance or extension appears to be required for cell immortality. Upregulation of telomerase expression also appears to be a characteristic of most tumor cells, which may contain chromosomes with relatively short telomeres (see, e.g., Pawelec, Mech. Aging Develop., 121: 181-185 (2000)). Nevertheless, the presence of telomerase in cancer cells appears to maintain telomeres of sufficient length to permit continuous generations of cell divisions. Thus, expression of telomerase has been viewed as a diagnostic marker for cancer cells, and the inhibition of telomerase activity or the repression of telomerase expression have been used as the bases for developing screens for anti-cancer drugs and possible anti-cancer therapies (see, e.g., U.S. Pat. No. 5,639,613; U.S. Pat. No. 5,770,613; U.S. Pat. No. 5,840,490; U.S. Pat. No. 5,863,936; U.S. Pat. No. 5,989,807).

From various studies, such as those described above, some molecules and methods for modulating expression of telomerase have been proposed. However, to date, there remains a need to develop acceptable and reliable means and methods to control the expression of telomerase in order to maintain and/or replenish telomeres that protect against the depletion or loss of genetic information from chromosomes during successive or prolong generations of cellular division and proliferation.

### SUMMARY OF THE INVENTION

The invention described herein solves the problem of shortening of telomeres and the attendant loss of genetic information from chromosomes during successive cellular divisions. In particular, this invention provides methods for upregulating expression of telomerase in eukaryotic cells, including mammalian cells, comprising contacting a cell, tissue, or organ with a peptide compound described herein. The methods may be used in a variety of applications, such as therapeutic and prophylactic treatments, diagnostic protocols, research methods, and drug screening procedures.

In one embodiment, the invention provides a method of upregulating telomerase expression in a eukaryotic cell, tissue, or organ, comprising contacting the eukaryotic cell, tissue, or organ with a peptide compound having the formula:

(SEQ ID NO:1)

R₁ Gln Tyr Lys Leu Gly Ser Lys Thr Gly Pro Gly Gln
R₂,

wherein R₁ is absent or is an amino terminal capping group and R₂ is absent or is a carboxy terminal capping group of the peptide compound; and wherein the peptide compound is present in an amount effective to upregulate expression of telomerase in the eukaryotic cell, tissues, or organ.

In another embodiment, the invention provides a method of upregulating telomerase expression in a eukaryotic cell, tissue, or organ, comprising contacting the eukaryotic cell, tissue, or organ with a peptide compound having the formula:

| | |
|---|---|
| R₁ Gln Thr Leu Gln Phe Arg R₂, | (SEQ ID NO:2) |

wherein R₁ is absent or is an amino terminal capping group and R₂ is absent or is a carboxy terminal capping group of the peptide compound; and wherein the peptide compound is present in an amount effective to upregulate expression of telomerase in the eukaryotic cell, tissue, or organ.

In yet another embodiment, the invention provides a method of upregulating telomerase expression in a eukaryotic cell, tissue, or organ, comprising contacting the eukaryotic cell, tissue, or organ with a peptide compound having the formula:

(SEQ ID NO:3)

R₁ Xaa₁ Gly Xaa₃ Xaa₄ Xaa₅ Xaa₆ Xaa₇ R2,

wherein Xaa₁ and Xaa₃ are, independently, aspartic acid or asparagine; R₁ is absent or is an amino terminal capping group of the peptide compound; Xaa₄ is absent or Gly; Xaa₅ is absent, Asp, or Phe; Xaa₆ is absent, Ala, or Phe; Xaa₇ is absent or Ala; R₂ is absent or is a carboxy terminal capping group of the peptide compound; and wherein the peptide compound is present in an amount effective to upregulate expression of telomerase in the eukaryotic cell, tissue, or organ. A particularly preferred method comprises contacting a eukaryotic cell, tissue, or organ with a peptide compound according to the above formula selected from the group consisting of:

| | |
|---|---|
| Asp Gly Asp, | |
| Asp Gly Asn, | |
| Asn Gly Asn, | |
| Asn Gly Asp, | |
| Asp Gly Asp Gly Asp, | (SEQ ID NO:4) |
| Asp Gly Asp Gly Phe Ala, | (SEQ ID NO:5) |
| Asp Gly Asp Gly Asp Phe Ala, | (SEQ ID NO:6) |
| Asp Gly Asn Gly Asp Phe Ala, | (SEQ ID NO:7) |
| Asn Gly Asn Gly Asp Phe Ala, | (SEQ ID NO:8) |
| and | |
| Asn Gly Asp Gly Asp Phe Ala, | (SEQ ID NO:9) |

wherein the peptide compound is present in an amount effective to upregulate expression of telomerase in the eukaryotic cell, tissue, or organ.

The invention also provides a method of upregulating telomerase expression in a eukaryotic cell, tissue, or organ, comprising contacting the eukaryotic cell, tissue, or organ with a peptide compound having the formula:

R₁ Asn Ser Thr R₂,

wherein R₁ is absent or is an amino terminal capping group; R₂ is absent or is a carboxy terminal capping group of the peptide compound; and wherein the peptide compound is present in an amount effective to upregulate expression of telomerase in the eukaryotic cell, tissue, or organ.

In yet another embodiment, the invention provides a method of upregulating telomerase expression in a eukaryotic cell, tissue, or organ, comprising contacting the eukaryotic cell, tissue, or organ with a peptide compound having the formula:

R₁ Phe Asp Gln R₂,

wherein R₁ is absent or is an amino terminal capping group; R₂ is absent or is a carboxy terminal capping group of the peptide compound; and wherein the peptide compound is present in an amount effective to upregulate expression of telomerase in the eukaryotic cell, tissue, or organ.

In another embodiment, the invention provides a method of upregulating telomerase expression in a eukaryotic cell, tissue, or organ, comprising contacting the eukaryotic cell, tissue, or organ with a peptide compound having the formula:

(SEQ ID NO:10)

R₁ Xaa₁ Xaa₂ Met Thr Leu Thr Gln Pro R₂,

wherein Xaa₁ is absent or Ser; Xaa₂ is absent or Lys; R₁ is absent or is an amino terminal capping group; R₂ is absent or is a carboxy terminal capping group of the peptide compound; and wherein the peptide compound is present in an amount effective to upregulate expression of telomerase in the eukaryotic cell, tissue, or organ. Particularly preferred is a method comprising contacting eukaryotic cell, tissue, or organ with a peptide compound selected from the group consisting of:

| | |
|---|---|
| Met Thr Leu Thr Gln Pro | (SEQ ID NO:11) |
| and | |
| Ser Lys Met Thr Leu Thr Gln Pro, | (SEQ ID NO:12) |

wherein the peptide compound is present in an amount effective to upregulate expression of telomerase in the eukaryotic cell, tissue, or organ.

In another embodiment, the invention provides a method of upregulating telomerase expression in a eukaryotic cell, tissue, or organ, comprising contacting the eukaryotic cell, tissue, or organ with a peptide compound having the formula:

| | |
|---|---|
| R₁ Asp Gly Xaa₃ Xaa₄ Xaa₅ R₂, | (SEQ ID NO:13) |

wherein R₁ is absent or is an amino terminal capping group; Xaa₃ is Glu or Leu; Xaa₄ is Ala or Glu; Xaa₅ is absent, Leu, or Ala; and R₂ is absent or is a carboxy terminal capping group of the peptide compound; and wherein the peptide compound upregulates expression of telomerase in the eukaryotic cell, tissue, or organ. A particularly preferred method of the invention for upregulating telomerase expression in a eukaryotic cell, tissue, or organ comprises contacting the eukaryotic cell, tissue, or organ with a peptide compound having the formula:

| | |
|---|---|
| [Ac] Asp Gly Glu Ala, | (SEQ ID NO:14) |

wherein [Ac] is an acetyl amino terminal capping group; and wherein the peptide compound is present in an amount effective to upregulate telomerase expression in the eukaryotic cell, tissue, or organ.

In another embodiment, the invention provides a method of upregulating telomerase expression in a eukaryotic cell, tissue, or organ, comprising contacting the eukaryotic cell, tissue, or organ with a peptide compound having the formula:

| | |
|---|---|
| R₁ Xaa₁ Xaa₂ Asp Gly Xaa₅ Xaa₆ Xaa₇ | (SEQ ID NO:15) |
| Xaa₈ Xaa₉ Xaa₁₀ Xaa₁₁ R₂; | |

wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or is any amino acid; Xaa₉ is absent or is any amino acid; Xaa₁₀ is absent or is any amino acid; Xaa₁₁ is absent or is any amino acid; and R₂ is absent or is a carboxy terminal capping group of the peptide compound; and wherein the peptide compound upregulates expression of telomerase in the eukaryotic cell, tissue, or organ.

The invention also provides a method of upregulating telomerase expression in a eukaryotic cell, tissue, or organ, comprising contacting the eukaryotic cell, tissue, or organ with a peptide compound having the formula:

R₁ Xaa₁ Xaa₂ Xaa₃ R₂,

wherein Xaa₁ is Asp, Asn, Glu, Gln, Thr, or Tyr; Xaa2 is absent or any amino acid (i.e., is variable); Xaa₃ is absent or is Glu, Thr, Ser, Gly, or Leu; R₁ is absent or is an amino terminal capping group and R₂ is absent or is a carboxy terminal capping group of the peptide compound; and wherein the peptide compound is present in an amount effective to upregulate expression of telomerase in the eukaryotic cell, tissue, or organ. A preferred embodiment of the method comprises the step of contacting a eukaryotic cell, tissue, or organ with a peptide compound of the above formula wherein Xaa₂ is selected from the group consisting of Val, Gly, Glu, and Gln. In another preferred embodiment, the method comprises contacting a eukaryotic cell, tissue, or organ with a peptide compound having an amino acid sequence selected from the group consisting of:

R₁ Asp Gly R₂, R₁ Asn Gly R₂, R₁ Glu Gly R₂, R₁
Gln Gly R₂,
and
R₁ Thr Val Ser R₂,

wherein R₁ is absent or is an amino terminal capping group and R₂ is absent or is a carboxy terminal capping group of the peptide compound; wherein the peptide compound is present in an amount effective to upregulate telomerase expression in the eukaryotic cell, tissue, or organ. A particularly preferred embodiment of the method comprises contacting a eukaryotic cell, tissue, or organ with the peptide compound R₁ Asp Gly; wherein R₁ is a thyronine amino terminal capping group, such as a monoiodo-, diiodo-, triiodo-, or tetraiodthyronine or a thyronine group having no iodine substitutions.

In still another embodiment, the invention provides a method of upregulating telomerase expression in a eukaryotic cell, tissue, or organ, comprising contacting the cell, tissue, or organ with a peptide compound having the formula:

R₁ Leu Xaa₂ Xaa₃ R₂,

wherein Xaa₂ is any amino acid; Xaa₃ is Gln or Tyr; R₁ is absent or is an amino terminal capping group; R₂ is absent or is a carboxy terminal capping group of the peptide compound; and wherein the peptide compound is present in an amount effective to upregulate expression of telomerase in the eukaryotic cell, tissue, or organ.

The invention also provides a method of upregulating telomerase expression in a eukaryotic cell, tissue, or organ, comprising contacting the eukaryotic cell, tissue, or organ with a peptide compound having the formula:

R₁ Met Thr Xaa₃ R₂,

wherein Xaa₃ is Asn, Asp, Glu, Thr, or Leu; R₁ is absent or is an amino terminal capping group; R₂ is absent or is a carboxy terminal capping group of the peptide compound; and wherein the peptide compound is present in an amount effective to upregulate expression of telomerase in the eukaryotic cell, tissue, or organ.

A peptide compound useful in the methods and compositions of the invention may contain one or more additional amino acids linked at the amino terminal and/or carboxy terminal amino acids of a “core sequence” of amino acids of the peptide compound, provided the peptide compound still is present in an amount effective to upregulate expression of telomerase in a cell, tissue, or organ. In a preferred embodiment, the invention provides a method of upregulating telomerase expression in a eukaryotic cell, tissue, or organ, comprising the step of contacting the eukaryotic cell, tissue, or organ with a peptide compound of any of the formulas described herein, wherein the peptide compound comprises an amino terminal capping group. An amino terminal capping group is linked, preferably covalently, to the amino terminal amino acid of the peptide of the peptide compound. Examples of amino terminal capping groups used in the invention include but are not limited to a lipoic acid moiety (Lip, in reduced or oxidized form); a glucose-3-O-glycolic acid moiety (Gga); 1 to 6 lysine residues; 1 to 6 arginine residues; a combination of 2 to 6 arginine and lysine residues; a thyronine group (e.g., a thyronine having no iodine substitutions or a mono-, di-, tri-, or tetraiodothyronine group); an acyl group of the formula R₃—CO—, where CO is a carbonyl group, and R₃ is a hydrocarbon chain having from 1 to 25 carbon atoms, and preferably 1 to 22 carbon atoms, and where the hydrocarbon chain may be saturated or unsaturated and branched or unbranched; and combinations thereof

An acyl group useful as an amino terminal capping group of a peptide compound employed in the methods and compositions of the invention preferably is an acetyl or a fatty acyl group. Even more preferably, the amino terminal capping group is an acyl group selected from the group consisting of acetyl, palmitoyl (Palm), and docosahexaenoly (DHA).

A thyronine group useful as an amino terminal capping group of a peptide compound employed in the invention may be selected from the group consisting of thyronine (i.e., having no iodine substitutions, “T₀”), a monoiodothyronine (“T₁”), a diiodothyronine (“T₂”), a triiodothyronine (“T₃”), and a tetrathyronine (“T₄”). A particularly preferred T4 group is a 3,5,3′,5′-tetraiodothyronine, which is also known as the thyroxin hormone. A thyronine group of a peptide compound useful in the invention may also be acylated (e.g., at the thyronine c-amino group with an acetyl group) or otherwise blocked, e.g., to prevent an undesired reaction with other molecules.

In another embodiment, the methods of the invention comprise a peptide compound, wherein the peptide compound has a carboxy terminal capping group linked, preferably covalently, to the peptide of the peptide compound. Particularly preferred are methods of the invention wherein the peptide compound has a carboxy terminal capping group that is a primary or secondary amine.

In addition to upregulating telomerase expression, the methods and compositions of the invention may also be used to upregulate one or more antioxidative enzymes (e.g., superoxide dismutase (SOD), catalase (CAT), and/or glutathione peroxidase (GPX)) that serve to protect or repair eukaryotic cells, tissues, and organs from damage by reactive oxygen species (ROS) and free radicals. Accordingly, methods and compositions of the invention may be employed to upregulate expression of telomerase and also one or more antioxidative enzymes in a eukaryotic cell, tissue, or organ, comprising the step of contacting the eukaryotic cell, tissue, or organ with a peptide compound described herein. Such methods are useful to prevent or counteract the damage or degeneration of eukaryotic cells, tissues, or organs due to disease, trauma, exposure to oxidative compounds, and/or the natural aging process.

The peptide compounds useful in the compositions and methods of the invention may also be prepared and used as one or more various salt forms, including acetate salts, depending on the needs for a particular composition or method.

Peptide compounds described herein may be administered to a eukaryotic individual (including a mammal, such as a human) to upregulate telomerase, and where desired one or more antioxidative enzymes, in the cells, tissues, or organs of the individual by any of a variety of routes including, but not limited to, oral (including sub-lingual) and parenteral routes. Parenteral routes of administration of peptide compounds in the methods of the invention include, without limitation, intravenous, intra-arterial, intramuscular, and subcutaneous routes.

The methods of the invention also provide improved methods of diagnosing cancer and other disease states associated with abnormal telomerase expression. In addition, the methods of the invention may be used in screening assays for compounds that inhibit telomerase activity or that down regulate (repress) expression of telomerase in cells.

Other uses of the methods of the invention include transplantation procedures, regenerative procedures for tissues and organs, and various research protocols in which it is desirable to upregulate telomerase expression in eukaryotic cells, tissues, or organs.

### BRIEF DESCRIPTION OF THE DRAWINGS

FIG. 1 shows diagrams of structures of representative thyronine groups (thyronyl) that may serve as amino terminal capping groups of peptide compounds used in the invention. Each structure shown in FIG. 1 is a form of a thyronine group that may be linked at its carbonyl carbon to the amino terminal amino group of a peptide. Some internal numbers (3, 5, 3′, 5′) are shown to indicate positions of iodine substitution in one or both phenyl groups of the thyronine group. “Thyroxin T₄” refers to the diagrammed tetraiodothyronine group, which is substituted with iodine at phenyl positions 3, 5, 3′, and 5′, and which is derived from the thyroxin hormone. “3,5,3′T₃” refers to the diagrammed triiodothyronine group, which is substituted with iodine at phenyl positions 3, 5, and 3′. “3,3′,5′rT₃” refers to the diagrammed triiodothyronine group, which is substituted with iodine at phenyl positions 3, 3′, and 5′. “T₀” refers to the diagrammed thyronine group, which has no iodine substitutions.

FIG. 2 shows an autoradiograph of results of a telomerase assay (telomeric repeat amplification protocol, “TRAP”, see text for details) of lysates from Nb2a cells isolated from cultures incubated in the presence or absence of peptide compound CMX-1 at a concentration of 100 μg/ml or of peptide compound CMX-3 at a concentration of 70 μg/ml for 6 hours (lanes 1-3) and 24 hours (lanes 4-6). Telomerase products formed by telomerase in lysates of cell cultures were separated on a polyacrylamide gel (1 μg of protein loaded per lane). Bracketed region of gel indicates location of telomerase products between 50 and 80 base pairs (bp). Lane 1, control cells (Ctrl) incubated 6 hours in absence of a peptide compound; lane 2, cells incubated with CMX-1 for 6 hours; lane 3, cells incubated with CMX-3 for 6 hours; lane 4, control cells incubated 24 hours in the absence of a peptide; lane 5, cells incubated with CMX-1 for 24 hours; lane 6, cells incubated with CMX-3 for 24 hours; lane M, positive marker control. Positive marker control represents the analysis of 1 μg of protein from a total cell lysate of immortalized HeLa cells.

FIGS. 3A and 3B. FIG. 3A shows an autoradiograph of results of a telomerase assay as in FIG. 2, except for rat brain primary cortical cell cultures, which were incubated for 4 hours in the absence of a peptide compound (lane 1); in the presence of CMX-1 at a concentration of 1 (lane 2), 10 (lane 3), or 70 μg/ml (lane 4); or in the presence of CMX-3 at a concentration of 1 (lane 5), 7 (lane 6), or 70 μg/ml (lane 7). Lane M, positive control, represents the analysis of 1 μg of protein from a total cell lysate of immortalized HeLa cells.

FIG. 3B shows a bar graph of the quantitative data obtained from each lane of the autoradiograph of FIG. 3A. Each bar graph correlates with a lane (concentration of a compound) in FIG. 3A. Positive control (Pos Ctrl) represents the analysis of 1 μg of protein from a total cell lysate of immortalized HeLa cells.

FIGS. 4A and 4B. FIG. 4A shows an autoradiograph of results of a telomerase assay of mouse brain tissue homogenates from 18-month old mice orally administered CMX-2 daily for 30 days at 0 mg/kg body weight (lanes 2 and 3); 0.03 mg/kg body weight (lanes 4 and 5); or 3.3 mg/kg of body weight; or from 5-month old mice (young mice) orally administered saline without peptide (lane 1). Lane M, positive control (Pos).

FIG. 4B shows a bar graph of the quantative data obtain from each lane of the autoradiograph of FIG. 4A. Each bar graph correlates with a lane of FIG. 4A.

FIGS. 5A and 5B. FIG. 5A shows an autoradiograph of results of a telomerase assay as in FIG. 3A, for rat brain primary cortical cell cultures, which were incubated for 24 hours in the absence of a peptide compound (lane 1) or in the presence of CMX-4 at a concentration of 1 ng/ml (lane 2). Lane Pos, positive control, represents the analysis of 1 μg of protein from a total cell lysate of immortalized HeLa cells.

FIG. 5B shows a bar graph of the quantitative data obtained from each lane of the autoradiograph of FIG. 5A. Each bar graph correlates with a lane (concentration of a compound) in FIG. 5A. Positive control (Pos) represents the analysis of 1 μg of protein from a total cell lysate of immortalized HeLa cells.

### DETAILED DESCRIPTION

In order that the invention may be better understood, the following terms are defined.

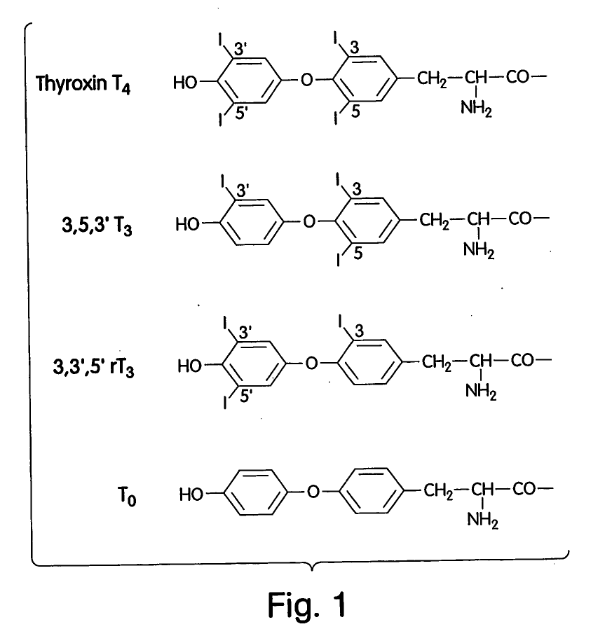

Abbreviations: Amino acid residues described herein may be abbreviated by the conventional three-letter or one-letter abbreviation know in the art (see, e.g., Lehninger, A. L., Biochemistry, second edition (Worth Publishers, Inc., New York, 1975), p. 72). Other abbreviations used herein include: “DHA” for the acyl form of docosahexaenoic acid moiety (i.e., docosahexaenoyl moiety); “Lip” for the acyl form of lipoic acid moiety; “Palm” for the acyl form of palmitic acid moiety (i.e., a palmitoyl group); “Ac” for the acyl form of acetic acid (i.e, an acetyl moiety); “Gga” for the acyl form of a glucose-3-O-glycolic acid moiety; “T₀” for a thyronine group having no iodine substitution; “T₁” for monoiodothyronine; “T₂” for diiodothyronine; “T₃” for triiodothryronine; and “T₄” for tetraiodothyronine. In addition, particular species of iodine-substituted thyronines may be abbreviated by listing the number of the position of one or both of the phenyl rings of thyronine that is substituted with an iodine atom. For example, “3′T₁” refers to a monoiodothyronine group substituted with an iodine at position 3′ of the terminal phenyl ring of thyronine, and “3,5 T₂” refers to a diiodothyronine group substituted with an iodine at positions 3 and 5 of the internal phenyl ring of thyronine (see, also FIG. 1). Still other abbreviations are indicated as needed elsewhere in the text

“Hydrocarbon” refers to either branched or unbranched and saturated or unsaturated hydrocarbon chains. Preferred hydrocarbon chains found in some of the peptide compounds described herein contain between 1 and 25. More preferred are hydrocarbon chains between 1 and 22 carbon atoms.

The term “polypeptide” refers to a linear polymer of two or more amino acid residues linked by peptide bonds, and the term “peptide” is used herein to refer to relatively short polypeptides, e.g., having fewer than about 20 amino acids.

“Peptide compound”, as understood and used herein, refers to any compound that contains at least one peptide bond. “Peptide compound” includes unmodified or underivatized peptides, typically containing fewer than about 20 amino acids, as well as derivatives of peptides. Derivative or derivatized peptides contain one or more chemical moieties other than amino acids that are covalently attached at the amino terminal amino acid residue, the carboxy terminal amino acid residue, or at an internal amino acid residue, for example, by a bond between a chemical moiety and a side chain of an internal amino acid residue of a peptide. Derivative peptide compounds useful in the methods of the invention also include any peptide conservative amino acid substitutions, addition of protective or capping groups on reactive moieties, and other changes that do not adversely destroy the activity of the peptide compound to upregulate expression of telomerase in a cell, tissue, or organ.

An “amino terminal capping group” of a peptide compound described herein is any chemical compound or moiety that is linked, preferably covalently, to the amino terminal amino acid residue of a peptide compound. An amino terminal capping group may be useful to inhibit or prevent intramolecular cyclization or intermolecular polymerization, to promote transport of a peptide compound across the blood-brain barrier, to prevent degradation of the peptide compound, or to provide a combination of these properties. A peptide compound that is useful in this invention and that possesses an amino terminal capping group may possess other beneficial activities as compared with the uncapped peptide compound, such as enhanced efficacy or reduced side effects. Examples of amino terminal capping groups of peptide compounds useful in the invention include, but are not limited to, 1 to 6 lysine residues, 1 to 6 arginine residues, a combination of arginine and lysine residues ranging from 2 to 6 residues, urethanes, urea compounds, a lipoic acid (“Lip”) or a palmitic acid moiety (i.e., palmitoyl group, “Palm”), glucose-3-O-glycolic acid moiety (“Gga”), a thyronine group (e.g., non-substituted thyronine, monoiodothyronine, diiodothyronine, triiodothyronine, tetraiodothyronine), and an acyl group that is covalently linked to the amino terminal amino acid residue of the peptide. Such acyl groups useful in the compositions of the invention may have a carbonyl group and a hydrocarbon chain that ranges from one carbon atom (e.g., as in an acetyl moiety) to up to 25 carbons (as in a docosahexaenoly moiety, “DHA”, which has a hydrocarbon chain that contains 22 carbons). Furthermore, the carbon chain of the acyl group may be saturated, as in a palmitoyl group, or unsaturated. It should be understood that when an acid or abbreviation for an acid (such as DHA, Palm, or Lip) is recited as an amino terminal capping group, the resultant peptide compound is the condensed product of the uncapped peptide and the acid.

A “carboxy terminal capping group” of a peptide compound described herein is any chemical compound or moiety that is linked, preferably covalently, to the carboxy terminal amino acid residue of the peptide compound. A carboxy terminal capping group may be useful to inhibit or prevent intramolecular cyclization or intermolecular polymerization, to promote transport of the peptide compound across the blood-brain barrier, to prevent degradation of the peptide compound, or to provide a combination of these properties. A peptide compound that is useful in the methods of this invention and that possesses a carboxy terminal capping group may possess other beneficial activities as compared with the uncapped peptide, such as enhanced efficacy, reduced side effects, enhanced hydrophilicity, or enhanced hydrophobicity. Carboxy terminal capping groups that are particularly useful in the peptide compounds described herein include primary or secondary amines that are linked by an amide bond to the a-carboxyl group of the carboxy terminal amino acid of the peptide compound. Other carboxy terminal capping groups useful in the invention include aliphatic primary and secondary alcohols and aromatic phenolic derivatives, including flavenoids, with C1 to C26 carbon atoms, which form esters when linked to the carboxylic acid group of the carboxy terminal amino acid residue of a peptide compound described herein.

“Effective amount” means an amount of a compound necessary to produce a desired effect An effective amount of a peptide compound in a method of the invention is the amount of a peptide compound that must be administered to cells, tissues, or an organ of an individual to produce an upregulation of the expression of telomerase in the cells, tissue, or organ. An effective amount of a compound for treating a disease state is any amount that when administered to an individual prevents (i.e., prophylactic treatment for) the development of a symptom of the disease or that eliminates or ameliorates (i.e., therapeutic treatment for) a symptom or manifestation of the disease.

“Isolated peptide compound” as used and understood herein means a peptide compound comprising a peptide as described herein and that is not present in a natural state, e.g., an isolated peptide compound is not present as part of a larger, naturally-occurring molecule, as a natural component of a biological source (e.g., cell, tissue, virus), or in unfractionated extract from a biological source. Isolated peptide compounds useful in the methods of the invention may be a purified, non-naturally occurring fragment of a naturally occurring protein or may be completely synthetic, i.e., having an amino acid sequence that is not found in nature and only produced using a peptide synthesis procedure.

“Pharmaceutical”, “pharmaceutically active compound”, and “pharmaceutical drug”, “drug”, and similar terms refer to any compound or composition that may be employed to treat a disease or condition in humans and/or other eukaryotic animals (e.g., in veterinary medicine). Such commonly known groups of pharmaceutically active compounds include, without limitation, anti-cancer compounds, antibiotics, anti-ulcer drugs, anti-viral drugs, immunostimulatory compounds, immunosuppressive compounds, psychotropic compounds (e.g., mood altering drugs), and anti-atherogenic compounds.

“Reactive oxygen species” or “ROS”, as understood and used herein, refers to any highly reactive and toxic oxygen-containing compound that may be generated in a cell in the course of normal electron transport system during respiration or that may be generated in a disease, during treatment with certain therapeutic agents for a particular disorder, or due to exposure to certain compounds. ROS include, but are not limited to, the superoxide anion (O₂.⁻) hydrogen peroxide (H₂O₂), singlet oxygen, lipid peroxides, and peroxynitrite.

“Free radical”, as understood and used herein, refers to any atom or any molecule or compound that possesses an odd (unpaired) electron. By this definition, the superoxide anion is also considered a negatively charged free radical. The free radicals of particular interest to this invention include highly reactive, highly oxidative molecules that are formed or generated in a eukaryotic cell during normal metabolism, in a eukaryotic cell having a diseased state, during treatment of a eukaryotic individual with therapeutic drugs (e.g., certain chemotherapeutic drugs), or due to exposure of a eukaryotic cell to certain compounds. Such free radicals are highly reactive and capable of causing oxidative damage to molecules, cells, tissues, and/or organs. One of the most common and potentially destructive types of the free radicals other than the superoxide anion is a hydroxyl radical. Typically, the generation of ROS, such as superoxide anion or singlet oxygen, also leads to one or more other harmful free radicals as well. Accordingly, phrases such as “ROS and free radicals” or “ROS and other free radicals”, as understood and used herein, are meant to encompass any or all of the entire population of highly reactive, oxidative molecular species or compounds that may be generated in a particular metabolic state or condition of cells, tissues, and organs of interest (see, for example, Somani et al, “Response of Antioxidant System To Physical and Chemical Stress,” In Oxidants. Antioxidants, and Free Radicals, chapter 6: 125-141 (Taylor & Francis, Washington, D.C., 1997)).

“Oxygen radical scavengers” or “lazaroids” are a class of compounds that have an ability to scavenge and detoxify ROS and free radicals. Vitamins A, C, E, and related antioxidant compounds, such as β-carotene and retinoids, are also members of this large class of compounds, as are antioxidative enzymes, such as superoxide dismutase (SOD), catalase (CAT), and glutathione peroxidase (GPX). In healthy individuals, sufficient levels of such antioxidative enzymes and other lazaroids are present both intracellularly and extracellularly to efficiently scavenge sufficient amounts of ROS and free radicals to avoid significant oxidative damage to cells, tissues, and organs.

“Upregulate” and “upregulation”, as used herein, refer to an elevation in the level of expression of a product of one or more genes in a cell or the cells of a tissue or organ. According to the invention, a peptide compound described herein will upregulate expression of a gene encoding telomerase beyond the levels normally found in cells or the cells of a tissue or organ that have not been treated (contacted) with the peptide compound. Thus, detection of an elevated level of telomerase enzyme activity, in the level of telomerase mRNA transcript for TERT, in the level of the telomerase RNA component (TR); in the level of TERT protein synthesis, or in the length or maintenance of a telomere are all examples of evidence of an upregulation of expression of telomerase. Thus, expression of one or both telomerase genes can be detected using any of a variety of protocols, including but not limited to, a telomerase enzyme assay, such as the assay described by Morin (Cell, 59: 521-529 (1989)) or the telomeric repeat amplification protocol (TRAP) of Piatsyzek (Methods in Cell Science, 17:1 (1995)); Northern blotting to detect an RNA transcript encoding the protein component (TERT) or the RNA component (TR) of telomerase; or Western inumunoblotting to detect the TERT protein component of telomerase gene. Preparations of purified human telomerase protein and antibodies to telomerase (e.g., rabbit anti-human TERT antibodies) useful in methods of detecting telomerase expression may be prepared by methods known in the art or obtained from a commercial source (e.g., Alpha Diagnostic International, Inc., San Antonio, Tex.).

Peptide compounds useful in the invention may also upregulate expression of key antioxidative enzymes employed by eukaryotic cells to protect against or repair oxidative damage, e.g., due to trauma, disease, exposure to exogenous or environmental oxidative compounds, and/or the aging process. Such antioxidative enzymes include, but are not limited to, superoxide dismutase (SOD), catalase (CAT), and glutathione peroxidase (GPX). An elevation in the level of SOD, CAT, or GPX mRNA transcript; in SOD, CAT, or GPX gene product (protein) synthesis; or in the level of SOD, CAT, and/or GPX enzyme activity indicate an upregulation of expression of the respective genes in cells, tissues, or organs. As with telomerase genes, expression of SOD, CAT, and GPX genes can be detected by a variety of ways, including but not limited to Northern blotting to detect mRNA transcripts encoding SOD, CAT, or GPX protein, by immunoblotting to detect synthesis of the gene product (protein), and standard enzyme assays for SOD, CAT, or GPX activity known in the art. Antibodies to SOD, CAT, and GPX may be obtained from a commercial source (e.g., Biogenesis, San Diego, Calif.) or by any of a variety of standard immunological procedures available in the art for producing antibodies to a given protein.

Any sample or collection of eukaryotic cells may be used in protocols described herein to detect upregulation of expression of telomerase and/or an antioxidative enzyme, including but not limited to, cells obtained from body fluids, such as blood; cells cultured in vitro; cells from biopsies; and tissues and organs containing cells of interest. Accordingly, any collection or group of eukaryotic cells is understood to be encompassed in the phrase “a eukayotic cell, tissue, or organ” and similar expressions.

Other terms will be evident as used in the following description.

Synthesis and Preparation of Peptide Compounds Useful in the Invention

The peptide compounds of the invention will upregulate telomerase expression in a eukaryotic cell, tissue, or organ activity when present at various concentrations, i.e., ranging from nanograms (ng) to milligram (mg) of peptide compound per milliliter (ml). Thus, in some cases, the potency is similar to that exhibited by various hormones, such as luteinizing hormone releasing hormone (LHRH) or human growth hormone. Accordingly, the peptide compounds described herein may be prepared, stored, and used employing much of the available technology already applied to the preparation, storage, and administration of known therapeutic hormone peptides.

The peptide compounds described herein may contain a peptide to which additional modifications have been made, such as addition of chemical moieties at the amino terminal and/or carboxy terminal amino acid residues of the peptide, conservative amino acid substitutions or modifications of side chains of internal amino acid residues of the peptide that do not destroy the desired activity of the peptide. It has been observed that intramolecular cyclization and some intermolecular polymerizations of peptides tend to inactivate or decrease the activity of the peptides. Accordingly, the most useful peptide compounds are the least susceptible to cyclization reactions or undesired polymerization or conjugation with other peptide compound molecules.

In addition to maintaining or enhancing the ability of peptide compounds described herein to upregulate expression of telomerase, some modifications may advantageously confer additional benefits. For example, amino terminal capping groups may promote transport of the peptide compound across the blood-brain barrier (“BBB”) (see, e.g., PCT publication WO 99/26620). This property is particularly important when a peptide compound is used to upregulate telomerase expression in brain tissue and parts of the central nervous system. Amino terminal capping groups that promote transport across the blood-brain barrier may also prevent cyclization of the peptide compound to which they are attached or may prevent polymerization with other peptide compounds.

Preferred amino terminal capping groups include a lipoic acid moiety (“Lip”), which can be attached by an amide linkage to the a-amino group of the amino terminal amino acid of a peptide. An amino terminally linked lipoic acid moiety may be in its reduced form where it contains two sulfhydryl groups or in its oxidized form in which the sulfhydryl groups are oxidized and form an intramolecular disulfide bond and, thereby, a heterocyclic ring structure. Another amino terminal capping group useful in preparing peptide compounds of the invention is a glucose-3-O-glycolic acid moiety (“Gga”), which can be attached in an amide linkage to the ax-amino group of the amino terminal amino acid of a peptide compound. The glucose moiety may also contain further modifications, such as an alkoxy group replacing one or more of the hydroxyl groups on the glucose moiety.

Another example of an amino terminal capping group useful in the peptide compounds described herein is an acyl group, which can be attached in an amide linkage to the α-amino group of the amino terminal amino acid residue of a peptide compound. The acyl group has a carbonyl group linked to a saturated or unsaturated (mono- or polyunsaturated), branched or unbranched, hydrocarbon chain of 1 to 25 carbon atoms in length, and more preferably, the hydrocarbon chain of the acyl group is 1 to 22 carbon atoms in length, as in DHA. The acyl group preferably is acetyl or a fatty acid. The fatty acid used as the acyl amino terminal capping group may contain a hydrocarbon chain that is saturated or unsaturated and that is either branched or unbranched. Preferably the hydrocarbon chain is 1 to 25 carbon atoms in length, and more preferably the length of the hydrocarbon chain is 1-22 carbon atoms in length. For example, fatty acids that are useful, in their corresponding acyl form, as amino terminal capping groups linked to the peptide compounds of this invention include, but are not limited to: caprylic acid (C8:0), capric acid (C10:0), lauric acid (C12:0), myristic acid (C14:0), palnitic acid (“Palm”) (C16:0), palmitoleic acid (C16:1), C16:2, stearic acid (C18:0), oleic acid (C18:1), vaccenic acid (C18:1-7), linoleic acid (C18:2-6), α-linolenic acid (C18:3-3), eleostearic acid (C18:3-5), β-linolenic acid (C18:3-6), C18:4-3, gondoic acid (C20: 1), C20:2-6, dihomo-γ-linolenic acid (C20:3-6), C20:4-3, arachidonic acid (C20:4-6), eicosapentaenoic acid (C20:5-3), docosenoic acid (C22: 1), docosatetraenoic acid (C22:4-6), docosapentaenoic acid (C22:5-6), docosapentaenoic acid (C22:5-3), docosahexaenoic acid (“DHA”) (C22:6-3), and nervonic acid (C24:1-9). Particularly preferred fatty acids used as acyl amino terminal capping groups for the peptide compounds described herein are a palmitoyl moiety (Palm) and a docosahexaenoyl moiety (DHA). DHA and, other fatty acyl groups also may promote transport of molecules to which they are linked across the blood-brain barrier (see, e.g., PCT publication WO 99/40112 and PCT publication WO 99/26620). Accordingly, such fatty acyl moieties are the of the type that is particularly preferred when a peptide compound described herein is administered to upregulate expression of telomerase in brain tissue and/or other parts of the central nervous system of an individual.

In addition, in certain cases the amino terminal capping group may be a lysine residue or a polylysine peptide, preferably where the polylysine peptide consists of two, three, four, five or six lysine residues. Longer polylysine peptides may also be used. Another amino terminal capping group that may be used in the peptide compounds described herein is an arginine residue or a polyarginine peptide, preferably where the polyarginine peptide consists of two, three, four, five, or six arginine residues, although longer polyarginine peptides may also be used. An amino terminal capping group of the peptide compounds described herein may also be a peptide containing both lysine and arginine, preferably where the lysine and arginine containing peptide is two, three, four, five or six residue combinations of the two amino acids in any order, although longer peptides that contain lysine and arginine may also be used. Lysine and-arginine containing peptides used as amino terminal capping groups in the peptide compounds described herein may be conveniently incorporated into whatever process is used to synthesize the peptide compounds to yield the derivatized peptide compound containing the amino terminal capping group.

Yet another example of an amino terminal capping group useful in the invention is a thyronine group (i.e., thyronyl). The α carboxyl group of a thyronine residue may be conjugated to an a amino group of an amino acid residue by condensation to form a peptide bond, e.g., during standard Merrifield synthesis of a peptide compound. Preferred thyronines useful as amino terminal capping groups of the peptide compounds described herein include but are not limited to thyronine (“T₀”), monoiodothyronine (“T₁”), diiodothyronine (“T₂”), triiodothyronine (“T₃”), and tetraiodothyronine (“T₄”). In the case of iodothyronines, a position that is substituted with iodine (iodinated) on one or both of the phenyl groups of a particular species of iodothyronine may vary, but preferably occurs at phenyl position 3, 5, 3′, 5′, or a combination thereof. Diagrams of several examples of thyronine groups useful in the invention are shown in FIG. 1 in a form in which each thyronine group may be linked at its carbonyl atom to another molecule (e.g., linked in a peptide bond to the amino terminal amino group of a peptide). The 3,5,3′, 5′-tetraiodothyronine group (3,5,3′,5′T₄) is derived from the thyroxin hormone (also referred to as “thyroxin T₄′). The α amino group of a thyronine may be free or linked to another moiety (e.g., blocked), as desired, using standard reactions known in the art, e.g., treatment with acetic anhydride may be used to couple an acetyl group to the αamino group of thyronine.

The peptide compounds useful in the methods of the invention may contain a carboxy terminal capping group. The primary purpose of this group is usually to prevent intramolecular cyclization or inactivating intermolecular crosslinking or polymerization. However, as noted above, a carboxy terminal capping group may provide additional benefits to a peptide compound described herein, such as enhanced efficacy, reduced side effects, and/or other desirable biochemical properties. An example of such a useful carboxy terminal capping group is a primary or secondary amine in an amide linkage to the carboxy terminal amino acid residue. Such amines may be added to the α-carboxyl group of the carboxy terminal amino acid of the peptide using standard amidation chemistry.

Cyclization, crosslinking, or polymerization of a peptide compound described herein may abolish all or so much of the activity of the peptide compound so that it cannot be used in the therapeutic or prophylactic compositions and methods of the invention.

In addition, peptide compounds described herein may contain one or more D-amino acid residues in place of one or more L-amino acid residues provided that the incorporation of the one or more D-amino acids does not abolish all or so much of the activity of the peptide compound that it cannot be used in the compositions and methods of the invention. Incorporating D-amino acids in place of L-amino acids may advantageously provide additional stability to a peptide compound, especially in vivo.

The peptide compounds can be made using standard methods or obtained from a commercial source. Direct synthesis of the peptides of the peptide compounds of the invention may be accomplished using conventional techniques, including solid-phase peptide synthesis, solution-phase synthesis, etc. Peptides may also be synthesized using various recombinant nucleic acid technologies, however, given their relatively small size and the state of direct peptide synthesis technology, a direct synthesis is preferred and solid-phase synthesis is most preferred.

In solid-phase synthesis, for example, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents, and reaction conditions used throughout the synthesis and are removable under conditions, which do not affect the final peptide product. Stepwise synthesis of the polypeptide is carried out by the removal of the N-protecting group from the initial (i.e., carboxy terminal) amino acid, and coupling thereto of the carboxyl end of the next amino acid in the sequence of the polypeptide. This amino acid is also suitably protected. The carboxyl group of the incoming amino acid can be activated to react with the N-terminus of the bound amino acid by formation into a reactive group such as formation into a carbodiimide, a symmetric acid anhydride, or an “active ester” group such as hydroxybenzotriazole or pentafluorophenyl esters.

The preferred solid-phase peptide synthesis methods include the BOC method, which utilizes tert-butyloxycarbonyl as the α-amino protecting group, and the FMOC method, which utilizes 9-fluorenyhnethloxycarbonyl to protect the α-amino of the amino acid residues, both methods of which are well-known by those of skill in the art (see, Stewart et al., In Solid-Phase Pentide Synthesis (W. H. Freeman Co., San Francisco 1989); Merrifield, J. Am. Chem. Soc., 85:2149-2154 (1963); (Bodanszky and Bodanszky, In The Practice of Peptide Synthesis (Springer-Verlag, New York 1984)). Amino terminal and carboxy terminal capping groups, if desired, may be added during or after peptide synthesis, depending on the specific moiety used as a capping group. For example, if the capping group is one or more amino acids, then such residues are simply incorporated into the protocol for synthesizing the peptide. If the capping group is not an amino acid, such as an acyl or amide group, it may be added after peptide synthesis using standard condensation or conjugation methods.

Peptide compounds described herein may also be prepared commercially by companies providing peptide synthesis as a service (e.g., BACHEM Bioscience, Inc., King of Prussia, PA; AnaSpec, Inc., San Jose, Calif.). Automated peptide synthesis machines, such as manufactured by Perkin-Elmer Applied Biosystems, also are available.

Peptide compounds useful in the methods of the invention may also be prepared and used in a salt form. Typically, a salt form of a peptide compound will exist by adjusting the pH of a composition comprising the peptide compound with an acid or base in the presence of one or more ions that serve as counter ion to the net ionic charge of the peptide compound at the particular pH. Various salt forms of the peptide compounds described herein may also be formed or interchanged by any of the various methods known in the art, including by using various ion exchange chromatography methods. Cationic counter ions that may be used in the compositions described herein include, but are not limited to, amines, such as ammonium ion; metal ions, especially monovalent or divalent ions of alkali metals (e.g., sodium, potassium, lithium, cesium), alkaline earth metals (e.g., calcium, magnesium, barium), transition metals (e.g., iron, manganese, zinc, cadmium, molybdenum), other metals (e.g., aluminum); and combinations thereof. Anionic counter ions that may be used in the compositions described herein include, but are not limited to, chloride, fluoride, acetate, trifluoroacetate, phosphate, sulfate, carbonate, citrate, ascorbate, sorbate, glutarate, ketoglutarate, and combinations thereof. Trifluoroacetate salts of peptide compounds described herein are typically formed during purification in trifluoroacetic acid buffers using high-performance liquid chromatography (HPLC). Trifluoroacetate salt forms of peptide compounds are generally not suited for ill vivo use, although they may be conveniently used in various in vitro cell culture studies or assays performed to test the activity or efficacy of a peptide compound of interest. The peptide compound may then be converted from the trifluoroacetate salt (e.g., by ion exchange methods) to or synthesized as a salt form that is acceptable for pharmaceutical use.

After being produced or synthesized, a peptide compound that is useful in the methods of the invention may be purified using methods known in the art. Such purification should provide a peptide compound of the invention in a state dissociated from significant or detectable amounts of undesired side reaction products; unattached or unreacted moieties used to modify the peptide compound; and dissociated from other undesirable molecules, including but not limited to other peptides, proteins, nucleic acids, lipids, carbohydrates, and the like. Standard methods of peptide purification may be employed to obtained isolated peptide compounds of the invention, including but not limited to various high-pressure (or performance) liquid chromatography (HPLC) and non-HPLC peptide isolation protocols, such as size exclusion chromatography, ion exchange chromatography, phase separation methods, electrophoretic separations, precipitation methods, salting in/out methods, immunochromatography, and/or other methods.

A particularly preferred method of isolating peptide compounds useful in compositions and methods of the invention employs reversed-phase HPLC using an alkylated silica column such as C₄—, C₈— or C₁₈-silica. A gradient mobile phase of increasing organic content is generally used to achieve purification, for example, acetonitrile in an aqueous buffer, usually containing a small amount of trifluoroacetic acid. Ion-exchange chromatography can also be used to separate peptide compounds based on their charge. The degree of purity of the peptide compound may be determined by various methods, including identification of a major large peak on HPLC. A peptide compound that produces a single peak that is at least 95% of the input material on an HPLC column is preferred. Even more preferable is a polypeptide that produces a single peak that is at least 97%, at least 98%, at least 99% or even 99.5% of the input material on an HPLC column.

In order to ensure that a peptide compound obtained using any of the techniques described above is the desired peptide compound for use in methods of the present invention, analysis of the compound's composition determined by any of a variety of analytical methods known in the art. Such composition analysis may be conducted using high resolution mass spectrometry to determine the molecular weight of the peptide. Alternatively, the amino acid content of a peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Since some of the peptide compounds contain amino and/or carboxy terminal capping groups, it may be necessary to remove the capping group or the capped amino acid residue prior to a sequence analysis. Thin-layer chromatography (TLC) methods may also be used to authenticate one or more constituent groups or residues of a desired peptide compound. Purity of a peptide compound may also be assessed by electrophoresing the peptide compound in a polyacrylamide gel followed by staining to detect protein components separated in the gel.

The various peptide compounds described herein are useful in the methods of the invention to upregulate telomerase expression and to prevent or counteract effects of loss of telomerase activity in cells and tissues, e.g., as occurs in the aging process (senescence), as well as may occur in various diseases, trauma, and various drug treatments, such as anti-cancer drug regimens that may otherwise undesirably destroy entire populations of cells of the circulatory or immune systems.

Preferred peptide compounds, excluding any amino and/or carboxy terminal capping group (i.e., the “core sequence”), are less than about 20 amino acids in length, and more preferably, less than 12 amino acids in length. Particularly preferred, are peptide compounds whose core sequences are less than 12 amino acids in length (see, e.g., peptide compound CMX-1 in Example 1), including those that are less than 10, amino acids, less than 9 amino acids, less than 8 amino acids, less than 7 amino acids, less than 6 amino acids, less than 5 amino acids (see, e.g., peptide compound CMX-2 in Example 1), less than 4 amino acids, and even less than 3 amino acids (see, e.g., dipeptide compounds CMX-3 and CMX-4 in Example 1) in length.

Methods of Upregulating Telomerase Expression

The invention provides a method of upregulating expression of telomerase in eukaryotic cell, tissue, or organ (including mammalian cells, tissues, or organs) by contacting the eukaryotic cell, tissue, or organ with a peptide compound described herein. The level of telomerase expression may be measured in any of a variety of samples including, but not limited to, cell lysates, cell extracts, tissue homogenates, biopsy samples, and sub-fractions thereof.

In one embodiment, the invention provides a method of upregulating telomerase expression in a eukaryotic cell, tissue, or organ, comprising contacting the eukaryotic cell, tissue, or organ with a peptide compound having the formula:

| | |
|---|---|
| R₁ Gln Tyr Lys Leu Gly Ser Lys Thr | (SEQ ID NO:1) |
| Gly Pro Gly Gln R₂, | |

wherein R₁ is absent or is an amino terminal capping group and R₂ is absent or is a carboxy terminal capping group of the peptide compound and wherein the peptide compound is present in an amount effective to upregulate expression of telomerase in the cells, tissue, or organ.

In another embodiment, the invention provides a method of upregulating telomerase expression in a eukaryotic cell, tissue, or organ, comprising contacting the eukaryotic cell, tissue, or organ with a peptide compound having the formula:

| | |
|---|---|
| R₁ Gln Thr Leu Gln Phe Arg R₂, | (SEQ ID NO:2) |

wherein R₁ is absent or is an amino terminal capping group and R₂ is absent or is a carboxy terminal capping group of the peptide compound and wherein the peptide compound is present in an amount effective to upregulate expression of telomerase in the cells, tissue, or organ.

The invention further provides a method of upregulating telomerase expression in a eukaryotic cell, tissue, or organ, comprising contacting the eukaryotic cell, tissue, or organ with a peptide compound having the formula:

| | |
|---|---|
| R₁ Xaa₁ Gly Xaa₃ Xaa₄ Xaa₅ Xaa₆ Xaa₇ | (SEQ ID NO:3) |
| R₂, | |

wherein Xaa₁ and Xaa₃ are, independently, aspartic acid or asparagine; R₁ is absent or is an amino terminal capping group of the peptide compound; Xaa₄ is absent or Gly; Xaa₅ is absent, Asp, or Phe; Xaa₆ is absent, Ala, or Phe; Xaa₇ is absent or Ala; R₂ is absent or is a carboxy terminal capping group of the peptide compound; and wherein the peptide compound is present in an amount effective to upregulate expression of telomerase in the cells, tissue, or organ. A particularly preferred method of upregulating expression of telomerase in a eukaryotic cell, tissue, or organ comprises contacting a eukaryotic cell, tissue, or organ with a peptide compound of having an amino acid sequence selected from the group consisting of:

| | |
|---|---|
| Asp Gly Asp, | |
| Asp Gly Asn, | |
| Asn Gly Asn, | |
| Asn Gly Asp, | |
| Asp Gly Asp Gly Asp, | (SEQ ID NO:4) |
| Asp Gly Asp Gly Phe Ala, | (SEQ ID NO:5) |
| Asp Gly Asp Gly Asp Phe Ala, | (SEQ ID NO:6) |
| Asp Gly Asn Gly Asp Phe Ala, | (SEQ ID NO:7) |
| Asn Gly Asn Gly Asp Phe Ala, | (SEQ ID NO:8) |
| and | |
| Asn Gly Asp Gly Asp Phe Ala. | (SEQ ID NO:9) |

The invention also provides a method of upregulating telomerase expression in a eukaryotic cell, tissue, or organ, comprising contacting the cell, tissue, or organ with a peptide compound having the formula:

R₁ Asn Ser Thr R₂,

wherein R₁ is absent or is an amino terminal capping group; R₂ is absent or is a carboxy terminal capping group of the peptide compound; and wherein the peptide compound is present in an amount effective to upregulate expression of telomerase in the cells, tissue, or organ.

In yet another embodiment, the invention provides a method of upregulating telomerase expression in a eukaryotic cell, tissue, or organ, comprising contacting the cell, tissue, or organ with a peptide compound having the formula:

R₁ Phe Asp Gln R₂,

wherein R₁ is absent or is an amino terminal capping group; R₂ is absent or is a carboxy terminal capping group of the peptide compound; and wherein the peptide compound is present in an amount effective to upregulate expression of telomerase in the cells, tissue, or organ.

In another embodiment, the invention provides a method of upregulating telomerase expression in a eukaryotic cell, tissue, or organ, comprising contacting the cell, tissue, or organ with a peptide compound having the formula:

(SEQ ID NO:10)

R₁ Xaa₁ Xaa₂ Met Thr Leu Thr Gln Pro R₂,

wherein Xaa₁ is absent or Ser; Xaa₂ is absent or Lys; R₁ is absent or is an amino terminal capping group; R₂ is absent or is a carboxy terminal capping group of the peptide compound; and wherein the peptide compound is present in an amount effective to upregulate expression of telomerase in the eukaryotic cell, tissue, or organ. Particularly preferred is a method of the invention to upregulate expression of a telomerase in a eukaryotic cell, tissue, or organ comprising contacting the eukaryotic cell, tissue, or organ with a peptide compound selected from the group consisting of:

| | |
|---|---|
| Met Thr Leu Thr Gln Pro | (SEQ ID NO:11) |
| and | |
| Ser Lys Met Thr Leu Thr Gln Pro. | (SEQ ID NO:12) |

In another embodiment, the invention provides a method of upregulating telomerase expression in a eukaryotic cell, tissue, or organ, comprising contacting the eukaryotic cell, tissue, or organ with a peptide compound having the formula:

| | |
|---|---|
| R₁ Asp Gly Xaa₃ Xaa₄ Xaa₅ R₂, | (SEQ ID NO:13) |

wherein R₁ is absent or is an amino terminal capping group; Xaa₃ is Glu or Leu; Xaa4 is Ala or Glu; Xaa₅ is absent, Leu, or Ala; and R₂ is absent or is a carboxy terminal capping group of the peptide compound; and wherein the peptide compound is present in an amount effective to upregulate expression of telomerase in the eukaryotic cell, tissue, or organ. A particularly method of upregulating telomerase expression in a eukaryotic cell, tissue, or organ comprises contacting the eukaryotic cell, tissue, or organ with a peptide compound having the formula:

| | |
|---|---|
| [Ac] Asp Gly Glu Ala, | (SEQ ID NO:14) |

wherein [Ac] is an acetyl amino terminal capping group; and the peptide compound is present in an amount effective to upregulate expression of telomerase in the eukaryotic cell, tissue, or organ.

The invention also provides a method of upregulating telomerase expression in a eukaryotic cell, tissue, or organ, comprising contacting the eukaryotic cell, tissue, or organ with a peptide compound having any of the following formulas:

| | |
|---|---|
| R₁ Asp Gly Glu Ala R₂, | (SEQ ID NO:14) |
| R₁ Asp Gly Glu Ala Leu R₂, | (SEQ ID NO:16) |
| R₁ Asp Gly Leu Glu Ala R₂, | (SEQ ID NO:17) |

wherein R₁ is absent or is an amino terminal capping group of the peptide compound and R₂ is absent or is a carboxy terminal capping group of the peptide compound; and wherein the peptide compound is present in an amount effective to upregulate expression of telomerase in the eukaryotic cell, tissue, or organ.

The invention also provides a method of upregulating telomerase expression in a eukaryotic cell, tissue, or organ, comprising contacting the eukaryotic cell, tissue, or organ with a peptide compound comprising a peptide, wherein the peptide has any of the following amino acid sequences:

| | |
|---|---|
| Asp Gly Glu Ala, | (SEQ ID NO:14) |
| Asp Gly Glu Ala Leu, | (SEQ ID NO:16) |
| Asp Gly Leu Glu Ala, | (SEQ ID NO:17) |

and wherein the peptide compound is present in an amount effective to upregulate expression of a gene encoding telomerase in the eukaryotic cell, tissue, or organ.

In another embodiment, the invention provides a method of upregulating telomerase expression in a eukaryotic cell, tissue, or organ, comprising contacting the eukaryotic cell, tissue, or organ with a peptide compound having the formula:

(SEQ ID NO:15)

R₁ Xaa₁ Xaa₂ Asp Gly Xaa₅ Xaa₆ Xaa₇ Xaa₈ Xaa₉
Xaa₁₀ Xaa₁₁ R₂;

wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or is any amino acid; Xaa₉ is absent or is any amino acid; Xaa₁₀ is absent or is any amino acid; Xaa₁₁ is absent or is any amino acid; and R₂ is absent or is a carboxy terminal capping group of the peptide compound; and wherein the peptide compound is present in amount effective to upregulate expression of telomerase in the eukaryotic cell, tissue, or organ.

Another method of the invention of upregulating telomerase expression in a eukaryotic cell, tissue, or organ, comprises contacting the eukaryotic cell, tissue, or organ with a peptide compound having the formula:

R₁ Xaa₁ Xaa₂ Xaa₃ R₂,

wherein Xaa₁ is Asp, Asn, Glu, Gln, Thr, or Tyr; Xaa₂ is absent or any amino acid (i.e., is variable); Xaa₃ is absent or is Glu, Thr, Ser, Gly, or Leu; R₁ is absent or is an amino terminal capping group and R₂ is absent or is a carboxy terminal capping group of the peptide compound; and wherein the peptide compound is present in an amount effective to upregulate expression of telomerase in the eukaryotic cell, tissue, or organ. This method may comprise contacting the eukaryotic cell, tissue, or organ with a peptide compound of the above formula wherein Xaa₂ is selected from the group consisting of Val, Gly, Glu, and Gln. For example, the method may comprise contacting a eukaryotic cell, tissue, or organ with a peptide compound having an amino acid sequence selected from the group consisting of:

R₁ Asp Gly R₂, R₁ Asn Gly R₂, R₁ Glu Gly R₂,
R₁ Gln Gly R₂,
and
R₁ Thr Val Ser R₂,

wherein R₁ is absent or is an amino terminal capping group and R₂ is absent or is a carboxy terminal capping group of the peptide compound; and wherein the peptide compound is present in an amount effective to upregulate telomerase expression in the eukaryotic cell, tissue, or organ. A particularly preferred example of this method comprises contacting a eukaryotic cell, tissue, or organ with a peptide compound having the formula R₁ Asp Gly, wherein R₁ is a thyronine amino terminal capping group, such as a monoiodo-, diiodo-, triiodo-, or tetraiodthyronine or a thyronine group having no iodine substitutions.

The invention also provides a method of upregulating telomerase expression in a eukaryotic cell, tissue, or organ, comprising the step of contacting the eukaryotic cell, tissue, or organ with a peptide compound having the formula:

R₁ Leu Xaa₂ Xaa₃ R₂,

wherein Xaa₂ is any amino acid; Xaa₃ is Gln or Tyr, R₁ is absent or is an amino terminal capping group; R₂ is absent or is a carboxy terminal capping group of the peptide compound; and wherein the peptide compound is present in an amount effective to upregulate expression of telomerase in the eukaryotic cell, tissue, or organ.

Another method of upregulating telomerase expression in a eukaryotic cell, tissue, or organ, comprises contacting the eukaryotic cell, tissue, or organ with a peptide compound having the formula:

R₁ Met Thr Xaa₃ R₂,

wherein Xaa₃ is Asn, Asp, Glu, Thr, or Leu; R₁ is absent or is an amino terminal capping group; R₂ is absent or is a carboxy terminal capping group of the peptide compound; and wherein the peptide compound is present in an amount effective to upregulate expression of telomerase in the eukaryotic cell, tissue, or organ.

Specific peptide compounds that may be used in methods to upregulate telomerase expression in a eukaryotic cell, tissue, or organ according to the invention include, but are not limited to, those peptide compounds having an amino acid sequence selected from the group consisting of:

(SEQ ID NO:1)

Gln Tyr Lys Leu Gly Ser Lys Thr Gly Pro Gly Gln,

(SEQ ID NO:2)

Gln Thr Leu Gln Phe Arg,

(SEQ ID NO:18)

Glu Thr Leu Gln Phe Arg,

(SEQ ID NO:19)

Gln Tyr Set Ile Gly Gly Pro Gln,

(SEQ ID NO:20)

Ser Asp Alg Ser Ala Alg Ser Tyr,

(SEQ ID NO:12)

Ser Lys Met Thr Leu Thr Gln Pro,

(SEQ ID NO:11)

Met Thr Leu Thr Gln Pro,

(SEQ ID NO:21)

Asp Gly Asp Gly Asp Phe Ala Ile Asp Ala Pro Glu,

(SEQ ID NO:6)

Asp Gly Asp Gly Asp Phe Ala,

(SEQ ID NO:4)

Asp Gly Asp Gly Asp,

(SEQ ID NO:8)

Asn Gly Asn Gly Asp Phe Ala,

(SEQ ID NO:22)

Asn Gly Asn Gly Asp,

(SEQ ID NO:7)

Asp Gly Asn Gly Asp Phe Ala,

(SEQ ID NO:23)

Asp Gly Asn Gly Asp,

(SEQ ID NO:9)

Asn Gly Asp Gly Asp Phe Ala,

(SEQ ID NO:24)

Asn Gly Asp Gly Asp,

(SEQ ID NO:25)

Asn Gly Asp Gly,

(SEQ ID NO:26)

Asp Gly Asp Gly Phe Ala,

(SEQ ID NO:27)

Asn Gly Asn Gly Phe Ala,

(SEQ ID NO:28)

Asp Gly Asn Gly Phe Ala,

(SEQ ID NO:29)

Asn Gly Asp Gly Phe Ala,

(SEQ ID NO:14)

Asp Gly Glu Ala,
Asp Gly Asp, Asn Gly Asn, Asp Gly Asn, Asn Gly
Asp, Asn Ser Thr, Phe Asp Gln, Met Thr Leu, Met
Thr Asp, Met Thr Asn, Met Thr Thr, Met Thr Glu,
Met Thr Gln, Thr Val Ser, Leu Thr Gln, Leu Thr
Gly, Leu Thr Tyr, Asp Gly, Asn Gly, Glu Gly, Gln
Gly, Glu Ala, Gln Ala, Gln Gly, Asp Ala, and Asn
Ala.

Peptide compounds useful in the methods and compositions of the invention may also contain one or more additional amino acids (e.g., one to six) linked at the amino terminal and/or carboxy terminal amino acids of a “core sequence” of any of the amino acid sequences described herein, and provided that the peptide compound is present in an amount effective to upregulate expression of telomerase as detected in a eukaryotic cell, tissue, or organ.

As noted above, a peptide compound having any amino acid sequence described herein and that is useful in the methods and compositions of the invention may also have an amino terminal capping group. For example, a preferred amino terminal capping group of peptide compounds used in the methods and compositions of the invention is selected from the group consisting of a lipoic acid moiety (Lip, in reduced or oxidized form); a glucose-3-O-glycolic acid moiety (Gga); 1 to 6 lysine residues; 1 to 6 arginine residues; a combination of arginine and lysine residues ranging from 2 to 6 residues; a thyronine group; an acyl group of the formula R₃—CO—, where CO is a carbonyl group, and R₃ is a hydrocarbon chain having from 1 to 25 carbon atoms, and more preferably 1 to 22 carbon atoms, and where the hydrocarbon chain may be saturated or unsaturated and branched or unbranched; and combinations thereof. More preferably, when a peptide compound used in a method of the invention has an acyl group as the amino terminal capping group, the acyl group is acetyl or a fatty acyl group. Even more preferably, the amino terminal capping group is an acyl group selected from the group of acyl groups derived from any of a variety of well known acids, including but not limited to, acetic acid (acetyl), palmitic acid (palmitoyl; “Palm”), and docosahexaenoic acid (docosahexaenoly; “DHA”).

A peptide compound having any amino acid sequence described herein and that is useful in the methods and compositions of the invention may also have a carboxy terminal capping group. Particularly preferred is a carboxy terminal capping group that is a primary or secondary amine.

Biological and Biochemical Activities

The methods of the invention employ peptide compounds that have the ability to upregulate telomerase expression in eukaryotic cells in vitro and in vivo, including cells of mammals such as humans, provided the cells contain at least one functional gene for each component of telomerase, i.e., for the protein component (TERT) and the RNA component (TR) of telomerase. A functional gene is one, which not only encodes a telomerase component, but also provides the necessary genetic information within and without the coding sequence for the encoded component so that transcription of the gene can occur. In the case of the TERT protein component, a functional TERT gene permits accurate synthesis of the mRNA transcript, which can be translated into a functional TERT protein component, which will associate with a TR RNA component to constitute a fully functional telomerase complex. In the case of the RNA component of telomerase, a functional TR gene permits accurate synthesis of the encoded RNA, which will associate with a TERT protein component to constitute a fully functional telomerase complex.

Advantageously, upregulation of telomerase expression in cells provides increased fidelity of genetic information on each chromosome in the cells throughout generations of mitotic divisions. Without wishing to be bound by theory, when the level of telomerase expression increases, it is believed that chromosomes are less susceptible to undesirable unraveling, bridging, and/or degradation from the ends of the chromosomes, thereby preventing loss of (i.e., preserving) critical genetic information on the chromosomes as cells divide or differentiate. Thus, maintenance of telomere length on chromosomes provides the functional genetic information required to maintain healthy cells, tissues, and organs that otherwise would degenerate due to disease, trauma, and the natural aging process that can occur in every eukaryotic species.

According to the invention, contacting eukaryotic cells in vitro or in vivo with a peptide compound described herein may result in at least about a 2-fold (and in increasing order of preference, at least about a 3-fold, 4-fold, 6-fold, or 8-fold) increase in the level of telomerase expression as compared to untreated cells. Desirable levels of preferred upregulation may be those that correspond to those present in healthy young animals, such as in a 4 month old individual in the case of mice or a 5, 10, 20, or even 50 year old individual in the case of humans, depending on the tissue of interest.

In addition, methods of the invention may not only upregulate telomerase but also one or more antioxidative enzymes (e.g., SOD, CAT, GPX) that repair and/or protect eukaryotic cells, tissues, and organs from oxidative damage.

Therapeutic and Prophylactic Methods

The correlation between the loss and/or degradation of telomeres of chromosomes to the progression of various disease states or age-related degenerative conditions is well established in the art. In general, a method or process that maintains healthy gene expression, such as a method for maintaining the length of telomeres to prevent loss of genetic material from chromosomes, is potentially capable of maintaining metabolic processes at healthy levels in cells that would otherwise deteriorate due to trauma, degenerative disease, or the natural aging process. Accordingly, the underlining concept of methods described herein is to upregulate telomerase expression so that enhanced telomerase activity will preserve and/or replenish telomeres on chormosomes to avoid loss of telomeres and the genetic material that they protect during cell division and proliferation (including activation of certain cell types), i.e., to preserve or reset to a healthy state the “telomeric clock” of each chromosome that would otherwise run down and halt further cell divisions.

A successful outcome to various surgical procedures performed on an individual, including grafts and transplantations of cells, tissues, or organs, may also be enhanced by the methods of the invention. For example, a peptide compound described herein may be administered to the individual before, during, or contemporaneously with a surgical procedure. For example, cells, tissues, or organs to be grafted or transplanted may be incubated in vitro with a peptide compound described herein, prior to grafting or transplanting to a recipient individual. In other cases, an ex vivo method of the invention may be required comprising the steps of extracting an individual's own cells or tissues (autologous cells or tissues), which express no or only depressed levels of telomerase; treating (e.g., by contacting, incubating, injecting) the extracted cells or tissues with a peptide compound to upregulate expression of telomerase therein; and re-introducing the treated cells or tissue back into the individual. In other situations, it may be useful or necessary to apply a peptide compound directly to cells, tissues, or organs, or to inject a peptide compound into an individual to upregulate expression of telomerase in cells, tissues, or organs of the individual in vivo or in situ.

The methods described herein to upregulate telomerase expression may be particularly useful to regenerate a cell population, tissue, and/or organ of an individual in need thereof as elevating levels of telomerase at one or more points in the regenerative process may help ensure complete proliferation and accurate differentiation of the cells into the desired cell population, tissue, or organ.

Procedures as described above may be applied to any of a variety of types of eukaryotic cells, tissues, or organs in which it is desirable to upregulate telomerase expression.

For example, stem cells can be treated according to the invention to upregulate telomerase expression to preserve the pluripotent compacity of these cells, which is particularly desirable when the stem cells are stored in tissue banks for future use. In addition, any tissue that serves as a source of stem cells, such as umbilical cord, may also be treated with a peptide compound according to the invention to preserve the pluripotency of the stem cells in the tissue source.

Particular cells and tissues that may be used in the methods described above and that may benefit from upregulation of telomerase expression include, but are not limited to, blood (e.g., for transfusions), stem cells, fibroblasts, chondrocytes, osteoblasts, osteocytes, pancreatic beta cells for producing insulin, neurons, glia cells, oligodendrocytes, bone marrow, skin grafts, T cells, killer T cells, other cells of the immune system, and combinations thereof. A variety of tissues, organs, or portions thereof, that may be transplanted or regenerated by various methods may also be treated with a peptide compound according to methods of the invention to upregulate telomerase expression and include, but are not limited to, bone, skeletal muscle, heart, smooth muscle, lung, liver, kidney, pancreas, veins, arteries, capillaries, eye, ear, and nose.

In addition, there are currently available a variety of methods, such as recombinant DNA methods, gene therapies, and transgenic methods, to express a therapeutically or otherwise desirable protein or nucleic acid in cells that are present in or are to be implanted into an individual in need thereof (see, e.g., Rudolph et al., Science, 287: 1253-1258 (2000), regarding adenoviral-mediated delivery of a TR gene to restore telomerase activity and inhibit liver cirrhosis). Accordingly, transformed and recombinantly modified cells are candidates for treatment with a peptide compound as described herein to upregulate expression of telomerase in order to preserve and maintain the telomeres of the chromosomes of such cells and thereby maintain the full genetic capability and expression of a desired gene(s) throughout cell proliferation and/or differentiation of the cells in an individual.

Cells, tissues, or organs of an individual may be treated with a peptide compound according to the invention to upregulate telomerase expression therein to counteract the degenerative effects of the aging process by replenishing and/or maintaining telomeres of chromosomes in cells to the length and number more characteristic of a younger and/or healthier individual. Accordingly, upregulating telomerase by the methods described herein is expected to prevent or inhibit a loss of critical metabolic and structural functions necessary for health and maintenance of cells, tissues, and/or organs of an individual that would otherwise occur over time in the absence of higher levels of telomerase expression. Thus, the methods of the invention may be used in anti-aging therapy and to prolong quality and length of life of an individual. In addition, such therapies are likely to be especially effective or enhanced by methods of the invention that also upregulate one or more antioxidative enzymes.

As noted above, Rudolph et al. (Science, 287: 1253-1258 (2000)) demonstrated adenoviral vector-mediated delivery of a TR gene to telomerase-deficient mice to restore telomerase activity, regenerate liver tissue in vivo, and alleviate liver cirrhosis. Thus, upregulating telomerase expression may be used to treat cirrhosis of the liver. Clearly, the loss of critical functions in cells, tissues, and/or organs of an individual may occur due to any of number of progressive, degenerative diseases, trauma, or the aging process. Accordingly, methods of the invention to upregulate telomerase expression may be used to treat (i.e., to prevent, improve, or reverse) a variety of conditions, including, but not limited to, liver cirrhosis, suppressed immune system, wrinkling skin, wound healing, scarring, burns, loss of muscle tone, muscular dystrophy, macular degeneration, myopia, far-sightedness, loss of body weight, malaise, lethargy, hair loss, loss of hair color, decreased expression of lactase, decreased sexual drive, decreased sexual function, male or female infertility, sleeplessness (including decreased rapid eye movement (REM)-phase sleep), life expectancy, and various types of neurodegenerative diseases. Progressive neurogenerative diseases that may be treated by methods described herein include, but are not limited to, Alzheimer's disease, Parkinson's disease, amyotrophic lateral sclerosis (ALS), and Tardive dyskinesia. Other progressive diseases that may be treated using methods of the invention are progressive, neuropsychiatric diseases, such as schizophrenia, may result from loss of normal expression of one or more gene products that may be critical for healthy brain function. Accordingly, as the methods described herein for upregulating telomerase expression may be useful in maintaining or reestablishing an adequate, healthy level of expression of one or more critical gene products, such methods may be used to treat or prevent any condition or progressive, degenerative disease that results from or is exasperated by such loss in expression of a critical gene product(s).

An effective treatment of a disease or condition of an individual is indicated by an amelioration, reversal, or prevention (prophylaxis) of development of one or more physical, mental, or biochemical symptoms thereof; including but not limited to, increased expression of a critical gene product(s) in cells, tissues, or organs; improved cell, tissue or organ function; improved mobility; improvement in healthy bone mass; improved muscle tone; improved appetite; improved connective tissue production; improved mental acuity or memory; improved emotional state; and enhanced life expectancy.

Research, Diagnostic, and Drug Screening Methods

The methods of the invention to upregulate expression of telomerase in cells may be applied to various research, diagnostic, and drug screening protocols.

The ability to upregulating telomerase expression in cells is now possible using methods described herein finds use in a research in a variety of fields, for example, cellular biology, molecular biology, immunology, developmental biology, and gerontology. The methods of the invention may be applied in vitro, ex vivo, in situ, and in vivo. Thus, a variety of studies may now be carried in which telomerase expression is upregulated to provide a variety of eukaryotic cells in which telomeres of the chromosomes are maintained and/or replenished.

The methods of the invention may also be incorporating into any of a variety of diagnostic methods for disease (particularly cancer) or toxic states to provide cells in which telomerase expression is upregulated. Cells in which expression of telomerase has been upregulated by methods described herein may be particularly useful as control cells, especially where a diagnostic method relies on detection of depressed or elevated levels of telomerase expression as a diagnosis of a disease or toxic state (see, e.g., U.S. Pat. No. 5,489,508; U.S. Pat. No. 5,639,613; U.S. Pat. No. 5,693,474; U.S. Pat. No. 5,840,490; U.S. Pat. No. 5,840,495; U.S. Pat. No. 5,989,807; U.S. Pat. No. 6,007,989).

Another application of the methods described herein is in screening for new drugs that modulate expression of telomerase. For example, screening protocols for an inhibitor of telomerase activity or a repressor of telomerase expression may be particularly effective using cells in which expression of telomerase is upregulated because a positive result means that the inhibitor or repressor function of a drug in the screen has overcome the elevated level of telomerase.

Accordingly, an example of a screening protocol for a test compound (drug) that inhibits telomerase activity or that represses telomerase expression in a eukaryotic cell (e.g., a mammalian cell) according to the invention may comprise the steps of:

(a) contacting a eukaryotic cell with a peptide compound described herein in an amount effective to upregulate telomerase expression in the eukaryotic cell;

(b) contacting the eukaryotic cell of step (a) with a test compound;

(c) assaying the level of telomerase activity or telomerase expression in the eukaryotic cell from step (b), wherein a decrease in telomerase activity or a decrease in telomerase expression in the eukaryotic cell indicates that said test compound is an inhibitor of telomerase activity or a repressor of telomerase expression.

Another use of cells, which have upregulated telomerase expression by methods of the invention, may be as a convenient, positive control, for example, where a protocol screens for a molecule other than the peptide compounds described herein that will upregulate telomerase expression.

Methods of the invention may also be employed to produce immortal cell lines, which require sufficient expression of telomerase to maintain telomeres indefinitely through generations of cell divisions. New immortalized cell lines produced using methods of the invention will find use in a variety of therapeutic, research, diagnostic, or screening protocols.

Research, diagnostic, and screening protocols comprising methods described herein may also employ any of a variety of assay or analysis formats, including but not limited to, microtiter plate assays; Western or other immunoblotting assays; Northern blotting for levels of RNA transcripts; cells immobilized on a finely divide, solid, dispersible substrate, such as beads or particles comprising cellulose, acrylamide, and/or agarose; cells immobilized on magnetic beads; microassay chips; surface plasmon resonance; fluorescence-activated cell sorter (FACS); capillary diffusion methods; and the like.

The methods described herein for upregulating telomerase expression in cells are effective at maintaining or replenishing telomere length and, thereby, the healthy expression of genetic material protected by telomeres. Of particular interest in this respect is the expression of receptor molecules that are critical for stimulating or suppressing various cellular processes. Receptors and the factors that modulate expression of such receptors are involved in a variety of cellular and systemic metabolic processes, especially those in multicellular eukaryotic organisms, such as mammals. Such receptor-mediated processes include, but are not limited to, signaling and cascade systems for gene expression, the immune response, endocrine regulation, nervous system function, tissue and organ regeneration, drug metabolism, maintenance of presser function, and memory. Accordingly, as the methods of the invention for upregulating telomerase expression maintain expression of critical gene products, such as receptors, such methods may be employed in procedures to screen for or identify genes encoding various receptor molecules, as well as, the genetic sequences and factors that may be involved in the expression of such receptor molecules.

Preparation of Pharmaceutical Compositions

Particularly preferred are methods of the invention that employ a peptide compound described herein in a pharmaceutical composition for administration to an individual to upregulate telomerase expression in cells, tissues, or organs of the individual. Accordingly, such methods of this invention comprise any of the isolated peptide compounds described herein, or pharmaceutically acceptable salts thereof, as the active ingredient (also called “pharmaceutical agent”) of a pharmaceutical composition. Pharmaceutical compositions employed in the invention may further comprise one or more other pharmaceutically acceptable ingredients, including an excipient (a compound that provides a desirable property or activity to the composition, but other than or in addition to that of the active ingredient), a carrier, an adjuvant, or a vehicle.

Pharmaceutical compositions of this invention can be administered to an individual, such as a mammal, and especially a human patient, in a manner similar to other therapeutic, prophylactic, and diagnostic agents, and especially compositions comprising therapeutic hormone peptides. The dosage to be administered, and the mode of administration will depend on a variety of factors including age, weight, sex, condition of the individual, and genetic factors, and will ultimately be decided by the attending physician or veterinarian. In general, dosage required for diagnostic sensitivity or therapeutic efficacy will range from about 0.001 to 25.0 mg/kg of host body mass (also referred herein as body weight).

Pharmaceutically acceptable salts of the peptide compounds useful in this invention include, for example, those derived from pharmaceutically acceptable inorganic and organic acids and bases. Examples of suitable acids include hydrochloric, hydrobromic, sulfuric, nitric, perchloric, fumaric, maleic, malic, pamoic, phosphoric, glycolic, lactic, salicylic, succinic, toluene-p-sulfonic, tartaric, acetic, citric, methanesulfonic, formic, benzoic, malonic, naphthalene-2-sulfonic, tannic, carboxymethyl cellulose, polylactic, polyglycolic, and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N—(C₁₋₄ alkyl)₄⁺ salts.

This invention also envisions the “quaternization” of any basic nitrogen-containing groups of a peptide compound disclosed herein, provided such quaternization does not destroy the ability of the peptide compound to upregulate expression of telomerase. Such quaternization may be especially desirable where the goal is to use a peptide compound containing only positively charged residues. For example, when charged amino acid residues are present in a peptide compound described herein, they are preferably either all basic (positively charged) or all acidic (negatively) which prevents formation of cyclic peptide compounds during storage or use. Cyclic forms of the peptide compounds may be inactive and potentially toxic. Thus, a quaternized peptide compound is a preferred form of a peptide compound containing basic amino acids. Even more preferred is the quaternized peptide compound in which the carboxy terminal carboxyl group is converted to an amide to prevent the carboxyl group from reacting with any free amino groups to form a cyclic compound. Any basic nitrogen can be quaternized with any agent known to those of skilled in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization or acids such as acetic acid and hydrochloric acid.

It should be understood that the peptide compounds described herein may be modified by appropriate functionalities to enhance selective biological properties, and in particular the ability to upregulate expression of telomerase. Such modifications are known in the art and include those, which increase the ability of the peptide compound to penetrate or being transported into a given biological system (e.g., brain, central nervous system, circulatory system, lymphatic system), increase oral availability, increase solubility to allow administration by injection, alter metabolism of the peptide compound, and alter the rate of excretion of the peptide compound. In addition, peptide compounds may be altered to a pro-drug form such that the desired peptide compound is created in the body of an individual as the result of the action of metabolic or other biochemical processes on the pro-drug. Such pro-drug forms typically demonstrate little or no activity in in vitro assays. Some examples of pro-drug forms may include ketal, acetal, oxime, and hydrazone forms of compounds, which contain ketone or aldehyde groups. Other examples of pro-drug forms include the hemi-ketal, hemi-acetal, acyloxy ketal, acyloxy acetal, ketal, and acetal forms.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions used in the methods of this invention may be administered by a variety of routes or modes. These include, but are not limited to, parenteral, oral, intratracheal, sublingual, pulmonary, topical, rectal, nasal, buccal, sublingual, vaginal, or via an implanted reservoir. Implanted reservoirs may function by mechanical, osmotic, or other means. The term “parenteral”, as understood and used herein, includes intravenous, intracranial, intraperitoneal, paravertebral, periarticular, periostal, subcutaneous, intracutaneous, intra-arterial, intramuscular, intra-articular, intrasynovial, intrasternal, intrathecal, and intralesional injection or infusion techniques. Such compositions are preferably formulated for parenteral administration, and most preferably for intravenous, intracranial, or intra-arterial administration. Generally, and particularly when administration is intravenous or intra-arterial, pharmaceutical compositions may be given as a bolus, as two or more doses separated in time, or as a constant or non-linear flow infusion.

The pharmaceutical compositions used in the invention may be in the form of a sterile injectable preparation, for example, as a sterile injectable aqueous or oleaginous suspension. Such a suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringers solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as those described in Pharmacoplia Halselica.

The pharmaceutical compositions of this invention may be orally administered in any orally acceptable dosage form including, but not limited to, aqueous solutions and suspensions, capsules, tablets, caplets, pills, oleaginous suspensions and solutions, syrups, and elixirs. In the case of tablets for oral use, carriers, which are commonly used include lactose and corn starch. Lubricating agents, such as magnesium stearate, are also typically added. For oral administration in a capsule form, useful diluents include lactose and dried cornstarch. Capsules, tablets, pills, and caplets may be formulated for delayed or sustained release.

When aqueous suspensions are to be administered orally, the peptide compound is advantageously combined with emulsifying and/or suspending agents. If desired, certain sweetening and/or flavoring and/or coloring agents may be added. Formulations for oral administration may contain 10%-95% (weight/volume, w/v) active ingredient, preferably 25%-70% (w/v). Preferably, a pharmaceutical composition for oral administration provides a peptide compound of the invention in a mixture that prevents or inhibits hydrolysis of the peptide compound by the digestive system, but allows absorption into the blood stream.

The pharmaceutical compositions of this invention may also be administered in the form of suppositories for vaginal or rectal administration. These compositions can be prepared by mixing a peptide compound described herein with a suitable non-irritating excipient, which is solid at room temperature but liquid at body temperature so that the composition will melt in a relevant body space to release the active ingredient. Such materials include, but are not limited to, cocoa butter, beeswax and polyethylene glycols. Formulations for administration by suppository may contain 0.5%-10% (w/v) active ingredient, preferably 1%-2% (w/v).

Topical administration of the pharmaceutical compositions used in the methods of the invention may be useful when the desired treatment involves areas or organs accessible by topical application, such as in wounds or during surgery. For application topically, the pharmaceutical composition may be formulated with a suitable ointment containing the active components suspended or dissolved in a carrier. Carriers for topical administration of the peptide compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the peptide compounds suspended or dissolved in a pharmaceutically suitable carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical composition may be formulated for topical or other application as a jelly, gel, or emollient, where appropriate. The pharmaceutical compositions used in this invention may also be topically applied to the lower intestinal tract by rectal suppository formulation or in a suitable enema formulation. Topical administration may also be accomplished via transdermal patches. This may be useful for maintaining a healthy skin tissue, promoting wound healing, decreasing scarring, and restoring skin damage (e.g., from burns).

The pharmaceutical compositions employed in this invention may be administered nasally, in which case absorption may occur via the mucus membranes of the nose, or inhalation into the lungs. Such modes of administration typically require that the composition be provided in the form of a powder, solution, or liquid suspension, which is then mixed with a gas (e.g., air, oxygen, nitrogen, etc., or combinations thereof) so as to generate an aerosol or suspension of droplets or particles. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Pharmaceutical compositions of the invention may be packaged in a variety of ways appropriate to the dosage form and mode of administration. These include but are not limited to vials, bottles, cans, packets, ampoules, cartons, flexible containers, inhalers, and nebulizers. Such compositions may be packaged for single or multiple administrations from the same container. Kits, of one or more doses, may be provided containing the composition in dry powder or lyophilized form; an appropriate diluent, which are to be combined shortly before administration; and instructions for preparation and/or administration of the reconstituted or otherwise prepared pharmaceutical composition. The pharmaceutical composition may also be packaged in single use pre-filled syringes, or in cartridges for auto-injectors and needleless jet injectors.

Multi-use packaging may require the addition of antimicrobial agents such as phenol, benzyl alcohol, meta-cresol, methyl paraben, propyl paraben, benzalconium chloride, and benzethonium chloride, at concentrations that will prevent the growth of bacteria, fungi, and the like, but be non-toxic when administered to a patient.

Consistent with good manufacturing practices, which are in current use in the pharmaceutical industry and which are well known to the skilled practitioner, all components contacting or comprising the pharmaceutical agent must be sterile and periodically tested for sterility in accordance with industry norms. Methods for sterilization include ultrafiltration, autoclaving, dry and wet heating, exposure to gases such as ethylene oxide, exposure to liquids, such as oxidizing agents, including sodium hypochlorite (bleach), exposure to high energy electromagnetic radiation, such as ultraviolet light, x-rays or gamma rays, and exposure to ionizing radiation.

Choice of method of sterilization will be made by the skilled practitioner with the goal of effecting the most efficient sterilization that does not significantly alter a desired biological function, such as, the ability to upregulate the level of telomerase expression of a peptide compound in a particular pharmaceutical composition. Ultrafiltration is a preferred method of sterilization for pharmaceutical compositions that are aqueous solutions or suspensions.

Details concerning dosages, dosage forms, modes of administration, composition and the like are further discussed in a standard pharmaceutical text, such as Remington's Pharmaceutical Sciences (1990), which is incorporated herein by reference.

As is well known in the art, structure and biological function of peptides are sensitive to chemical and physical environmental conditions such as temperature, pH, oxidizing and reducing agents, freezing, shaking and shear stress. Due to this inherent susceptibility to degradation, it is necessary to ensure that the biological activity of a peptide compound used as a pharmaceutical agent be preserved during the time that the agent is manufactured, packaged, distributed, stored, prepared and administered by a competent practitioner. Many technical approaches have been developed to stabilize pharmaceutical proteins, polypeptides, or peptide molecules so as to preserve their biological potency and efficacy, and such stabilizing techniques may be applied to peptide compounds of the compositions and methods of the invention, including:

a) Freeze-drying and lyophilization (refer to Carpenter et al., Pharm. Res., 14(8): 969 (1997), incorporated by reference);

b) Addition of “stabilizers” to the aqueous solution or suspension of the peptide or protein. For example, U.S. Pat. No. 5,096,885 discloses addition of glycine, mannitol, pH buffers, and the non-ionic surfactant polysorbate 80 to human growth hormone as means to stabilize the protein during the process of filtration, vial filling, and cold storage or lyophilization; U.S. Pat. No. 4,297,344 discloses stabilization of coagulation factors II and VIII, antithrombin III and plasminogen against heat by adding selected amino acids and a carbohydrate; U.S. Pat. No. 4,783,441 discloses a method for prevention of denaturation of proteins such as insulin in aqueous solution at interfaces by the addition of surface acting substances, within a particular pH range; and U.S. Pat. No. 4,812,557 discloses a method of stabilizing interleukin-2 using human serum albumin;

c) Freeze/thaw methods wherein the peptide compound is mixed with a cryoprotectant and stored frozen at very low temperatures (e.g., −70° C.);

d) Cold, non-frozen storage (e.g., less than 4° C.), optionally with a cryoprotectant additive such as glycerol;

e) Storage in a vitrified, amorphous state, e.g., as described in U.S. Pat. No. 5,098,893;

f) Storage in a crystalline state; and

g) Incorporation into liposomes or other micelles.

Additional aspects of the invention will be further understood and illustrated in the following examples. The specific parameters included in the following examples are intended to illustrate the practice of the invention and its various features, and they are not presented to in any way limit the scope of the invention.

### EXAMPLES

### Example 1

### Synthesis of Representative Peptide Compounds Useful in Methods of the Invention

Representative peptide compounds useful in the methods of the invention were synthesized by solid phase Merrifield synthesis (Merrifield, J. Am. Chein. Soc., 85:2149-2154 (1963)) and characterized as indicated below.

CMX-1: a peptide compound that is a polypeptide having the amino acid sequence Asp Gly Asp Gly Asp Phe Ala Ile Asp Ala Pro Glu (SEQ ID NO:21),

CMX-2: a peptide compound having the formula [Ac] Asp Gly Glu Ala (SEQ ID NO: 14),

CMX-3: a peptide compound that is a dipeptide having the amino acid sequence Asp Gly, and

CMX-4: a peptide compound having the formula R₁ Asp Gly, wherein R₁ is the iodine-substituted thyronine amino terminal capping group, 3,5,3′-triiodothyronine (3,5,3′T₃ shown in FIG. 1).

Amino terminal capping groups, as indicated by the bracketed groups “[DHA]-”, “[Lip]-”, and “[Ac]-”, which represent an all cis-docasahexaenoic moiety, a lipoic acid moiety, and an acetyl moiety, respectively, could be attached to the α-amino group of the amino terminal amino acid residue of the indicated peptide compounds (Shashoua and Hesse, Life Sci. 58:1347-1357 (1996)). The T₃ iodothyronine group was attached by condensation to form a peptide bond with the α-amino group of the amino terminal aspartic acid residue in the CMX-4 peptide compound during Merrifield synthesis (see, below).

The peptide compounds were synthesized using standard procedures. Briefly, the peptides were synthesized using the solid phase Merrifield process (Merrifield, J Am. Chem. Soc., 85:2149-2154 (1963)). This method allows the synthesis of a peptide of a specific amino acid sequence bound on a polymeric resin. Each newly synthesized peptide was then released from the resin by treating with trifluoroacetic acid (TFA). The resultant trifluoroacetic acid peptide salt was purified by ether precipitation according to standard procedures (see, Groos and Meienhofer, In The peptides, analysis, synthesis, biology, vol.2 (Academic Press, New York 1983)).

For amino terminal substituted peptides (i.e., peptides containing an acyl amino terminal capping group), each peptide was synthesized with blocked side chains using solid phase Merrifield synthesis (see above). The bound peptide was then treated with an equimolar amount of an anhydride of one of the following acids: acetic acid, DHA, or lipoic acid, in the presence of 4-dimethylamino pyridine under argon atmosphere. The reaction was carried out for about three hours to obtain Amino terminal coupling. Evidence of complete Amino terminal coupling was obtained prior to peptide isolation. This was established by monitoring the ninhydrin staining properties of the resin bound peptides using standard procedures (Kaiser et al., Anal. Biochem., 34: 595-598 (1970)). The Amino terminal coupled (capped) peptide molecule was then released from the resin by treatment with TFA and purified by precipitation with cold ether followed by HPLC using methanolic HCl (50:50) as the eluant.

The final peptide products were white solids after lyophilization. Structures were confirmed by amino acid analyses, by migration as a single peak on HPLC, and molecular weight determinations by mass spectrometry. For most uses, it was essential to completely remove TFA from the peptide compound. This was achieved by repeated dissolution of the peptide in glacial acetic acid followed by concentration in vacuo in rotary evaporator. Complete absence of TFA was established by mass spectrometry.

### Example 2

### Telomerase Activity Assay: Telomeric Repeat Amplification Protocol (TRAP)

Telomerase activity was assayed in human cells and tumors by telomeric repeat amplification protocol (TRAP) (see, Piatsyzek, Methods in Cell Science, 17:1 (1995)). Whole cell lysates were prepared from approximately 10⁶ Nb2a cells. Cells were washed once with PBS, repelleted, and 20 μl of lysate buffer (10 mM Tris-HCl pH 7.5, 1 mM MgCl₂, 1 mM EGTA, 0.1 mM benzamidine, 5 mM β-mercaptoethanol, 0.5% CHAPS, 10% glycerol) was added directly to the washed pellets. The suspensions were incubated on ice for 30 minutes to allow cell swelling and lysis. Lysates were cleared by centrifugation at 12,000×g for 20 minutes at 4° C. Total protein concentration was determined for the cleared supernatant using a Coomassie Reagent protocol (Pierce). Supernatants were stored at −70° C.

Telomerase activity was measured in total cellular lysates using a telomeric repeat amplification protocol (TRAP). DNA oligomer “TS” 18-mer (AATCCGTCGAGCAGAGTT) (SEQ ID NO:30) was end-labeled with P³² in a 20 μl reaction containing 50 mM Tris-HCl (pH 7.5), 10 mM MgCl₂, 0.1 mM EDTA, 1 mM DTT, 1 pmol TS primer, 1 pmol [γ-³²P]-ATP (4,500 Ci/mmol), and 5 U polynucleotide kinase (Ambion). The reaction was incubated for 20 minutes at 37° C., and then stopped by incubating for 5 minutes at 85° C. Labeled TS oligomer was stored at −20° C. until use.

Telomerase-induced hexamer extensions to the TS primer were performed in a 50 μl reaction volume containing 20 mM Tris-HCl (pH 8.3), 1.5 mM MgCl₂, 63 mM KCl, 0.05% Tween-20, 1 mM EGTA, 50 μM dNTPs, 0.1 pmol ³²P-labeled TS primer, 1 pmol reverse primer RP (CTAACCCTAACCCTAACC) (SEQ ID NO:31), 1.0 μg cell lysate, and 2 U Taq polymerase (Ambion). The reaction was incubated for 30 minutes at 30° C. Hexamer extensions were then amplified by PCR using the following program: denaturation at 94° C. for 30 seconds, annealing at 59° C. for 30 seconds, 27 cycles denaturation/annealing. Radiolabeled ladders were separated by electrophoresis on 10% polyacrylamide gels, dried onto 3 MM paper, and visualized by autoradiography with Kodak X-Omat AR film. Exposure times averaged 4 hours at 25° C. Ladders were quantitated by excision from the gel, and counting in a Bench Count (Beckman).

### Example 3

### Upregulation of Telomerase in Neuroblastoma Cells Treated with CMX-1 and CMX-3

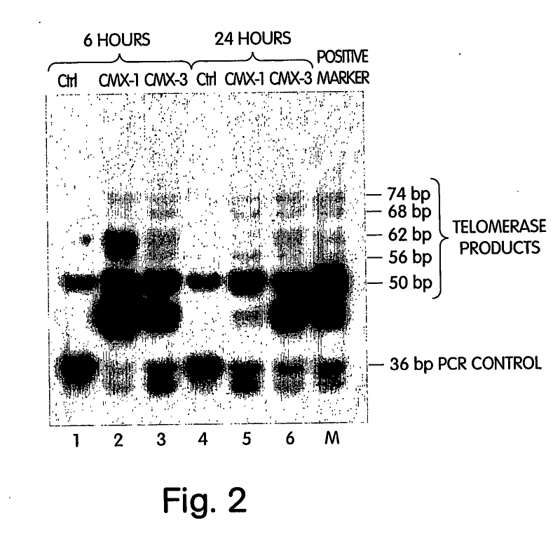

Mouse neuroblastoma cells (NB2a/DL) were grown in cultures in Delbecco's modified Eagle medium, supplemented with 10% fetal calf serum (FCS) in P-75 culture flasks in an atmosphere containing air and 5% CO₂ at 37° C. as described in Comell-Bell et al. (1990). P-75 culture flasks containing confluent monolayers of neurons in culture were incubated with peptide CMX-1 (100 μg/ml) or peptide CMX-3 (70 μg/ml) for 6 and 24 hours. Cytoplasmic proteins were then isolated and analyzed for upregulation of telomerase using the telomeric repeat amplification protocol (TRAP) described above. According to standard TRAP assay procedure, telomerase activity was detected as a P³²-labeled DNA, which was separated by gel electrophoresis and found to correspond to a ladder series of DNA hexamers for the 50-80 base pair (bp) regions (see, FIG. 2). The control cultures that received no peptide treatment showed no upregulation of telomerase activity using TRAP. These results show that telomerase was upregulated in the neuroblastoma cell line over control levels when treated with either of the peptide compounds.

### Example 4

### Upregulation of Telomerase in Primary Cortical Rat Brain Cell Cultures Treated with CMX-1 and CMX-3

The ability of representative peptide compounds described herein to upregulate telomerase was also tested in primary cortical cultures of rat brain using the TRAP telomerase assay described above.

Cultures containing mixtures of neurons and glia were grown to confluence for 12 days from newborn rat primary cortical cells as described above according to the method of Comell-Bell et al. (1990) in the presence of CX-1 or CMX-3. Negative control cultures were not exposed to a peptide compound during the growth period. Positive control (“Pos” or “Pos Ctrl”) represents the analysis of 1 μg of protein from a total cell lysate of immortalized HeLa cells.

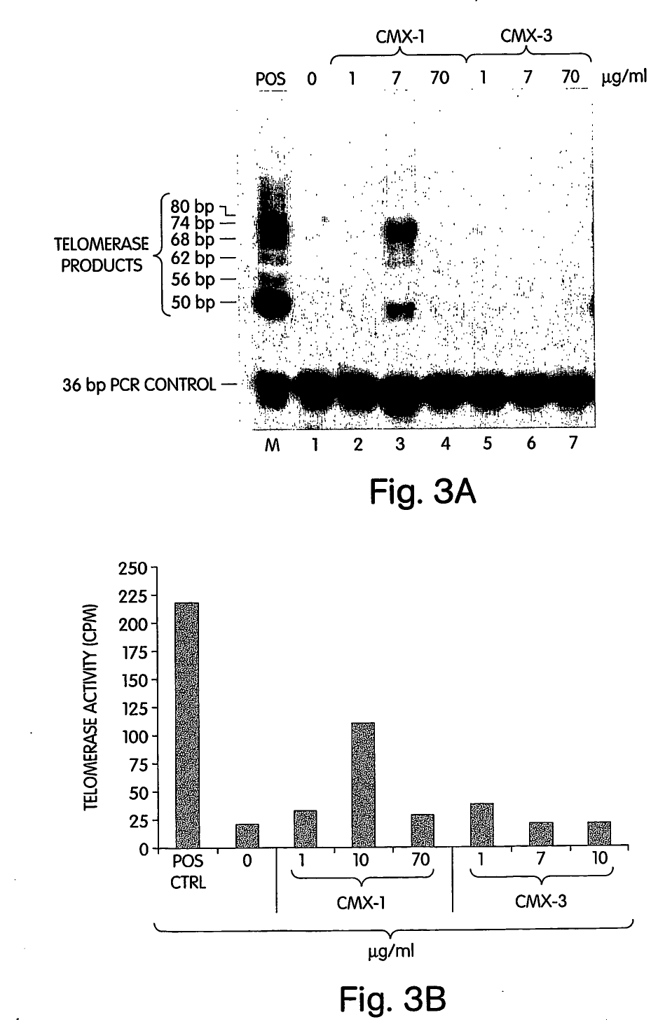

The results (see FIGS. 3A and 3B) showed that both CMX-1 and CMX-3 upregulated expression of telomerase in the primary rat brain cortical cultures. In this case, the P³²-labeled DNA precursors were incorporated into the series of hexamer base pairs showing the upregulation of the telomerase. The total radioactivity corresponding to base pairs ladders from 50 to 80 bp was measured by counting the particular region of the gel. Dose-response studies showed that treatment of cells with CMX-1 at a concentration of 10 μg/ml resulted in a greater than 4-fold increase in the level of telomerase expression relative to control levels. CMX-3 showed maximum effects at a dose of 1 μg/ml (i.e., 2-fold upregulation over control). The CMX-1 peptide compound gave an inverted U-shape dose-response curve. The highest concentrations of peptide compound tested produced increasingly less upregulation of the enzyme, as is typically observed at some point along most dose response curves.

### Example 5

### Upregulation of Telomerase in vivo by Treatment with CMX-2

The ability of peptide compounds described herein to upregulate telomerase in old and young mice was also studied. Here, the old mice (17 months old) were treated daily by gavage for 30 days with an oral dose of peptide CMX-2 in saline at doses of 0, 0.03, 0.3, and 3.3 mg of peptide compound per kilogram (kg) of body weight. Control animals received saline only without peptide compound. Animals were sacrificed by decapitation after 30 days and dissected to isolate the brain, heart, and liver, which was frozen at −70° C. for subsequent analysis. Each tissue was thawed, and then homogenized in a dounce homogenizer using ten volumes of homogenizer buffer (10 mM Tris-HCl, pH 7.5, 1 mM MgCl₂, 1 mM EGTA, 0.1 mM benzamidine, 5 mM β-mercaptoethanol, 0.5% CHAPS, 10% glycerol) to obtain a total cell lysate. Lysates were centrifuged at 14,000×g for 5 minutes at 4° C. The supernatant was harvested to yield a cleared cytoplasmic fraction.

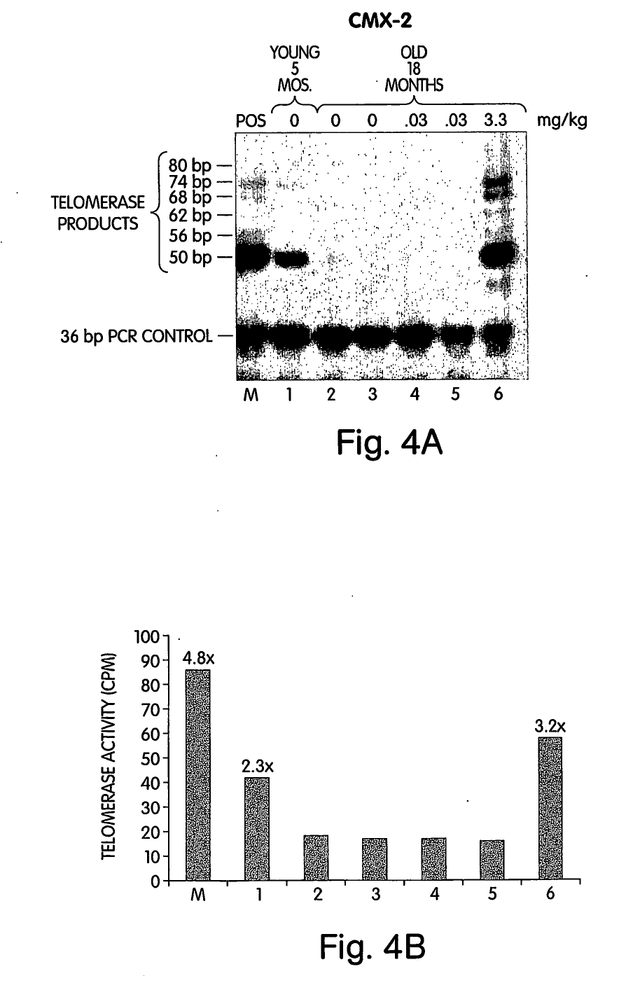

Telomerase activity was then measured in the cytoplasmic protein fraction of tissue homogenates. The results of assays of the brain tissue homogenates (see FIGS. 4A and 4B) showed that old animals (18 months old) had barely detectable telomerase activity. Measurements of brain tissue homogenates from two separate, untreated, old mice showed low baseline activity, whereas those of untreated young mice (control 5 months old) had a 2.3-fold higher activity. Brain tissue homogenates of old animals that received a dose of 3.3 mg of CMX-2 per kg body weight showed a high telomerase activity, which was 3.2-fold higher than that obtained from untreated controls.

Similar results were observed with the heart tissue homogenates from mice in this study.

The results showed that telomerase activity can be increased to a level higher in the old animals as compared to young (5 months old) mice. These results confirmed that the peptide compound CMX-2 upregulated telomerase in both brain and heart tissues, which normally do not have any significant amount of telomerase.

The above experiments demonstrated that an amino terminal acetyl dipeptide compound (CMX-3), a tetrapeptide (CMX-2) and a twelve amino acid residue peptide (dodecapeptide) (CMX-1) can upregulated the endogenous telomerase of mammalian cells and tissues in vitro and in vivo.

### Example 6

### Upregulation of Telomerase and Antioxidative Enzymes in Primary Cortical Rat Brain Cell Cultures Treated with a Triiodothyronyl Dipeptide Compound (CMX-4)

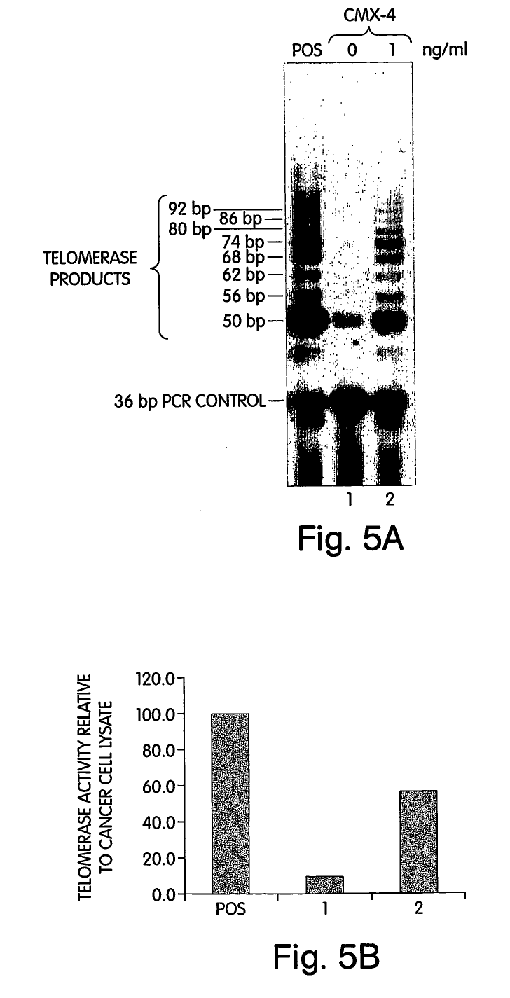

Primary cortical rat brain cell cultures were prepared as described above in Example 4 except that cells were cultured following a 24-hour exposure to 0 or 1 nanograms (ng)/ml of CMX-4. The results (FIGS. 5A and 5B) showed that relatively low amounts of CMX-4 were effective at upregulating telomerase in primary cortical rat brain cells. Positive control (“Pos”) represents the analysis of 1 μg of protein from a total cell lysate of immortalized HeLa cells.

In addition, the levels of antioxidative enzymes superoxide dismutase (SOD), catalase (CAT), and glutathione peroxidase (GPX) were determined using a standard immunoblotting protocol (see, Adams et al., J. Cell. Biochem., 77: 221-233 (2000)).

The results showed that, as with telomerase expression, all three antioxidative enzymes was upregulated in the rat brain cells compared to untreated control cells (data not shown) using the same nanogram amounts of the CMX-4 peptide compound.

The results of this study indicated that CMX-4 is a relatively potent compound for upregulating expression of telomerase as well as key antioxidative enzymes that protect a cell against oxidative damage.

Other variations and embodiments of the invention described herein will now be apparent to those of ordinary skill in the art without departing from the scope or spirit of the invention.

All patents, applications, and publications cited in the above text are incorporated herein by reference.

1-31. (canceled)

32. A method of upregulating telomerase expression in a eukaryotic cell, tissue, or organ derived from a mammal in need thereof, comprising contacting the eukaryotic cell, tissue, or organ with an effective amount of at least one peptide compound wherein the peptide compound contains fewer than 20 amino acids and more than 1 amino acids.

33. The method of claim 32, wherein the peptide compound contains fewer than 9 amino acids.

34. The method of claim 32, wherein the peptide compound contains two amino acids.

35. The method of claim 32, wherein the peptide compound comprises an amino terminal capping group.

36. The method of claim 32, wherein the peptide compound comprises a carboxy terminal capping group.

37. The method of claim 32, wherein the peptide compound is selected from the group consisting of:

| | |
|---|---|
| R₁ Gln Tyr Lys Leu Gly Ser Lys Thr | (SEQ ID NO:1) |
| Gly Pro Gly Gln R₂, | |
| R₁ Gln Thr Leu Gln Phe Arg R₂, | (SEQ ID NO:2) |

wherein R₁ is absent or is an amino terminal capping group and R₂ is absent or is a carboxy terminal capping group of the peptide compound.

38. The method of claim 32, wherein the peptide compound having the formula:
(SEQ ID NO:3)
R₁ Xaa₁ Gly Xaa₃ Xaa₄ Xaa₅ Xaa₆ Xaa₇ R₂,
wherein Xaa₁ and Xaa₃ are, independently, aspartic acid or asparagine; R₁ is absent or is an amino terminal capping group of the peptide compound; Xaa₄ is absent or Gly; Xaa₅ is absent, Asp, or Phe; Xaa₆ is absent, Ala, or Phe; Xaa₇ is absent or Ala; R₂ is absent or is a carboxy terminal capping group of the peptide compound.

39. The method of claim 32, wherein the peptide compound is selected from the group consisting of

| | |
|---|---|
| Asp Gly Asp, | |
| Asp Gly Asn, | |
| Asn Gly Asn, | |
| Asn Gly Asp, | |
| Asp Gly Asp Gly Asp, | (SEQ ID NO:4) |
| Asp Gly Asp Gly Phe Ala, | (SEQ ID NO:5) |
| Asp Gly Asp Gly Asp Phe Ala, | (SEQ ID NO:6) |
| Asp Gly Asn Gly Asp Phe Ala, | (SEQ ID NO:7) |
| Asn Gly Asn Gly Asp Phe Ala, | (SEQ ID NO:8) |
| and | |
| Asn Gly Asp Gly Asp Phe Ala. | (SEQ ID NO:9) |

40. The method of claim 32, wherein the peptide compound is represented by the following formula:
R₁ Asn Ser Thr R₂,
wherein R₁ is absent or is an amino terminal capping group; R₂ is absent or is a carboxy terminal capping group.

41. The method of claim 32, wherein the peptide compound is represented by the following formula:
R₁ Phe Asp Gln R₂,
wherein R₁ is absent or is an amino terminal capping group; R₂ is absent or is a carboxy terminal capping group.

42. The method of claim 32, wherein the peptide compound is represented by the following formula:
(SEQ ID NO:10)
R₁ Xaa₁ Xaa₂ Met Thr Leu Thr Gln Pro R₂,
wherein Xaa₁ is absent or Ser; Xaa₂ is absent or Lys; R₁ is absent or is an amino terminal capping group; R₂ is absent or is a carboxy terminal capping group.

43. The method of claim 32, wherein the peptide compound is selected from the group consisting of

| | |
|---|---|
| Met Thr Leu Thr Gln Pro | (SEQ ID NO:11) |
| and | |
| Ser Lys Met Thr Leu Thr Gln Pro. | (SEQ ID NO:12) |

44. The method of claim 32, wherein the peptide compound is represented by the following formula:

| | |
|---|---|
| R₁ Asp Gly Xaa₃ Xaa₄ Xaa₅ R₂, | (SEQ ID NO:13) |

wherein R₁ is absent or is an amino terminal capping group; Xaa₃ is Glu or Leu; Xaa4 is Ala or Glu; Xaa₅ is absent, Leu, or Ala; and R₂ is absent or is a carboxy terminal capping group of the peptide compound.

45. The method of claim 32, wherein the peptide compound is selected from the group consisting of:

| | |
|---|---|
| R₁ Asp Gly Glu Ala R₂, | (SEQ ID NO:14) |
| R₁ Asp Gly Glu Ala Leu R₂, | (SEQ ID NO:16) |
| and | |
| R₁ Asp Gly Leu Glu Ala R₂, | (SEQ ID NO:17) |

wherein R₁ is absent or is an amino terminal capping group of the peptide compound and R₂ is absent or is a carboxy terminal capping group of the peptide compound.

46. The method of claim 45, wherein the peptide compound is represented by the following formula:
[Ac] Asp Gly Glu Ala,
wherein [Ac] is an acetyl amino terminal capping group.

47. The method of claim 32 wherein the peptide compound is represented by the following formula:
(SEQ ID NO:15)
R₁ Xaa₁ Xaa₂ Asp Gly Xaa₅ Xaa₆ Xaa₇ Xaa₈ Xaa₉
Xaa₁₀ Xaa₁₁ R₂,
wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or is any amino acid; Xaa₉ is absent or is any amino acid; Xaa₁₀ is absent or is any amino acid; Xaa₁₁ is absent or is any amino acid; and R₂ is absent or is a carboxy terminal capping group.

48. The method of claim 32, wherein the peptide compound is represented by the following formula:
R₁ Xaa₁ Xaa₂ Xaa₃ R₂,
wherein Xaa₁ is Asp, Asn, Glu, Gln, Thr, or Tyr; Xaa₂ is absent or any amino acid; Xaa₃ is absent or is Glu, Thr, Ser, Gly, or Leu; R₁ is absent or is an amino terminal capping group and R₂ is absent or is a carboxy terminal capping group of the peptide compound.

49. The method of claim 48, wherein Xaa₂ is selected from the group consisting of Val, Gly, Glu, and Gln.

50. The method of claim 32, wherein the peptide compound is selected from the group consisting of:
R₁ Asp Gly R₂,
R₁ Asn Gly R₂,
R₁ Glu Gly R₂,
R₁ Gln Gly R₂,
and
R₁ Thr Val Ser R₂,
wherein R₁ is absent or is an amino terminal capping group and R₂ is absent or is a carboxy terminal capping group of the peptide compound.

51. The method of claim 32, where the peptide compound has the formula:
R₁ Asp Gly,
wherein R₁ is a thyronine group.

52. The method according to claim 51, wherein the thyronine group is selected from the group consisting of a thyronine group having no iodine substitutions, a monoiodothyronine, a diiodothyronine, a triiodothyronine, and a tetraiodothyronine.

53. The method according to claim 51, wherein the thyronine group is triiodothyronine.

54. The method according to claim 53, wherein the triiodothyronine is 3,5,3′-triiodothyronine.

55. The method of claim 32, wherein the peptide compound has the formula:
R₁ Leu Xaa₂ Xaa₃ R₂,
wherein Xaa₂ is any amino acid; Xaa₃ is Gln or Tyr; R₁ is absent or is an amino terminal capping group; R₂ is absent or is a carboxy terminal capping group of the peptide compound.

56. The method of claim 32, wherein the peptide compound has the formula:
R₁ Met Thr Xaa₃ R₂,
wherein Xaa₃ is Asn, Asp, Glu, Thr, or Leu; R₁ is absent or is an amino terminal capping group; R₂ is absent or is a carboxy terminal capping group of the peptide compound.

57. The method of claim 35, wherein the amino terminal capping group is selected from the group consisting of a lipoic acid moiety (Lip); a glucose-3-O-glycolic acid moiety (Gga); 1 to 6 lysine residues; 1 to 6 arginine residues; a combination of 2 to 6 lysine and arginine residues; a thyronine group; an acyl group of the formula R₃—CO—, where CO is a carbonyl group and R₃ is a hydrocarbon chain having from 1 to 25 carbon atoms; and combinations thereof.

58. The method of claim 35, wherein the amino terminal capping group is an acyl group of the formula R₃—CO—, where CO is a carbonyl group and R₃ is a hydrocarbon chain having from 1 to 22 hydrocarbons and wherein the hydrocarbon chain is a saturated, unsaturated, branched, or unbranched hydrocarbon chain.

59. The method of claim 35, wherein the amino terminal capping group is an acyl group.

60. The method of claim 59, wherein the acyl group is a fatty acyl group.

61. The method of claim 59, wherein the acyl group is selected from the group consisting of: acetyl, palmitoyl (Palm), and docosahexaenoly (DHA).

62. The method of claim 57, wherein the thyronine group is selected from the group consisting of a thyronine having no iodine substitutions, a monoiodothyronine, a diiodothyronine, a triiodothyronine, and a tetraiodothyronine.

63. The method of claim 32, wherein the method is an ex vivo method.

64. The method of claim 63, further comprising obtaining cell, tissue or organ from the mammal.

65. A method of treating a disease or condition caused by or relating to telomerase activity in a human patient in need thereof, comprising contacting the cells, tissue or organ derived from the patient with an effective amount of at least one peptide compound wherein the peptide compound contains fewer than 20 amino acids and more than 1 amino acids.

66. The method of claim 65, wherein the disease or condition is liver cirrhosis.

67. The method of claim 65, wherein the disease or condition is organ transplant.

68. The method of claim 65, wherein the method is an ex vivo method.

69. The method of claim 65, wherein the peptide compound is administered through a route selected from the group consisting of an oral route, an intravenous route, an intra-arterial route, an intramuscular route, and a subcutaneous route.
